(19) United States
(12) Patent Application Publication
(10) Pub. No.: US 2013/0317281 A1
(43) Pub. Date: Nov. 28, 2013

(54) TRANSCRANIAL MAGNETIC STIMULATION FOR IMPROVED ANALGESIA

(71) Applicants: M. Bret SCHNEIDER, Portola Valley, CA (US); David J. MISHELEVICH, Playa del Rey, CA (US); John W. SADLER, Belmont, CA (US)

(72) Inventors: M. Bret SCHNEIDER, Portola Valley, CA (US); David J. MISHELEVICH, Playa del Rey, CA (US); John W. SADLER, Belmont, CA (US)

(21) Appl. No.: 13/888,263

(22) Filed: May 6, 2013

Related U.S. Application Data
(63) Continuation-in-part of application No. 13/877,428, filed on Jun. 27, 2013.
(60) Provisional application No. 61/391,552, filed on Oct. 8, 2010.
(60) Provisional application No. 61/642,975, filed on May 4, 2012.

(51) Int. Cl. A61N 2/00

(52) U.S. Cl. CPC A61N 2/008
USPC 600/13; 600/9

(57) ABSTRACT

Described herein are methods for neuromodulating brain activity of one or more target brain regions, the methods using Transcranial Magnetic Stimulation (TMS) to produce robust analgesia. In particular, described herein are systems for arranging one or more (e.g., a plurality) of TMS electromagnets in a configuration and applying sufficient energy to neuromodulate the dorsal anterior cingulate gyrus relative to cortical brain regions to significant modulate pain, including the pain of fibromyalgia.

### CROSS REFERENCE TO RELATED APPLICATIONS

This patent application claims priority as a continuation-in-part of U.S. patent application Ser. No. 13/877,428, filed Apr. 2, 2013, and titled “TRANSVERSE TRANSCRANIAL MAGNETIC STIMULATION COIL PLACEMENT FOR IMPROVED ANALGESIA,” which claims the benefit under 35 U.S.C. 371 to International Patent Application No. PCT/US2011/055594, filed Oct. 10, 2011, and titled “TRANSVERSE TRANSCRANIAL MAGNETIC STIMULATION COIL PLACEMENT FOR IMPROVED ANALGESIA,” Publication No. WO 2012/048319, which claims priority to U.S. Provisional Patent Application No. 61/391,552, filed Oct. 8, 2010. All of these patent applications are herein incorporated by reference in their entirety.

This patent application also claims priority to U.S. Provisional Patent Application No. 61/642,975, filed May 4, 2012, and titled “REPETITIVE TRANSCRANIAL MAGNETIC STIMULATION SYSTEM AND METHODS OF USE,” which is herein incorporated by reference in its entirety.

This patent application may be related to one or more of the following patents and pending patent applications (U.S. and PCT applications), each of which is herein incorporated by reference in its entirety: U.S. Pat. No. 7,520,848, titled “ROBOTIC APPARATUS FOR TARGETING AND PRODUCING DEEP, FOCUSED TRANSCRANIAL MAGNETIC STIMULATION,” issued on Apr. 21, 2009; U.S. patent application Ser. No. 12/402,404, titled “ROBOTIC APPARATUS FOR TARGETING AND PRODUCING DEEP, FOCUSED TRANSCRANIAL MAGNETIC STIMULATION,” filed on Mar. 11, 2009, Publication No. US-2009-0234243-A1; U.S. patent application Ser. No. 11/429,504, titled “TRAJECTORY-BASED DEEP-BRAIN STEREOTACTIC TRANSCRANIAL MAGNETIC STIMULATION,” filed on May 5, 2006, U.S. Pat. No. 8,052,591; U.S. patent application Ser. No. 12/669,882, titled “DEVICE AND METHOD FOR TREATING HYPERTENSION VIA NON-INVASIVE NEUROMODULATION,” filed on Jan. 20, 2010, Publication No. US-2010-0256436-A1; U.S. patent application Ser. No. 12/671,260, titled “GANTRY AND SWITCHES FOR POSITION-BASED TRIGGERING OF TMS PULSES IN MOVING COILS,” filed on Jan. 29, 2010, Publication No. US-2010-0256439-A1; U.S. patent application Ser. No. 12/670,938, titled “FIRING PATTERNS FOR DEEP BRAIN TRANSCRANIAL MAGNETIC STIMULATION,” filed on Jan. 27, 2010, Publication No. US-2010-0256438-A1; U.S. patent application Ser. No. 12/677,220, titled “FOCUSED MAGNETIC FIELDS,” filed on Mar. 9, 2010, Publication No. US-2010-0331602-A1; International Application No. PCT/US2008/077851, titled “SYSTEMS AND METHODS FOR COOLING ELECTROMAGNETS FOR TRANSCRANIAL MAGNETIC STIMULATION,” filed on Sep. 26, 2008, Publication No. WO 2009/042863; International Application No. PCT/US2008/081048, titled “INTRA-SESSION CONTROL OF TRANSCRANIAL MAGNETIC STIMULATION,” filed on Oct. 24, 2008, Publication No. WO 2009/055634; U.S. patent application Ser. No. 12/324,227, titled “TRANSCRANIAL MAGNETIC STIMULATION OF DEEP BRAIN TARGETS,” filed on Nov. 26, 2008, U.S. Pat. No. 8,267,850; International Application No. PCT/US2009/045109, titled “TRANSCRANIAL MAGNETIC STIMULATION BY ENHANCED MAGNETIC FIELD PERTURBATIONS,” filed on May 26, 2009, Publication No. WO 2009/143503; U.S. patent application Ser. No. 12/185,544, titled “MONOPHASIC MULTI-COIL ARRAYS FOR TRANSCRANIAL MAGNETIC STIMULATION,” filed on Aug. 4, 2008, Publication No. US-2009-0099405-A1; U.S. patent application Ser. No. 12/701,395, titled “CONTROL AND COORDINATION OF TRANSCRANIAL MAGNETIC STIMULATION ELECTROMAGNETS FOR MODULATION OF DEEP BRAIN TARGETS,” filed on Feb. 5, 2010, Publication No. US-2010-0185042-A1; International Application No. PCT/US2010/020324, titled “SHAPED COILS FOR TRANSCRANIAL MAGNETIC STIMULATION,” filed on Jan. 7, 2010, Publication No. WO 2010/080879; and U.S. patent application Ser. No. 12/838,299 TRANSCRANIAL MAGNETIC STIMULATION FIELD SHAPING, filed on Jul. 16, 2010, Publication No. US-2010-0286470-A1.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are transcranial magnetic stimulation (TMS), including repetitive transcranial magnetic stimulation (rTMS), systems and methods for modulating brain targets so as to produce analgesia using low-frequency Transcranial Magnetic Stimulation (TMS).

### BACKGROUND

Repetitive transcranial magnetic stimulation (rTMS) involves placing an electromagnetic coil on the scalp while high-intensity current is rapidly turned on and off in the coil through the discharge of capacitors. This produces a time-varying magnetic field that lasts for about 100 to 200 microseconds. The magnetic field is typically about 2 Tesla. The proximity of the brain to the time-varying magnetic field results in current flow in neural tissue. Thus, rTMS provides a powerful opportunity for non-invasive stimulation of superficial cerebral cortex in both healthy subjects and those with a range of psychiatric or neurological disorders. Primarily, however, rTMS stimulation studies have focused on the stimulation superficial cortex, and observing secondary effects in deeper regions of the brain. This is because conventional TMS device designs have been unable to focally modulate subcortical regions directly without overwhelming superficial cortex.

In the early 1990s, Mark George and colleagues described the antidepressant effect of rTMS when applied to the left dorsolateral prefrontal cortex. Since that time, rTMS has become a recognized as an effective method for treating depression. One rTMS device (NeuroStar system by Neuronetics Inc, Malvern, Pa.) has received FDA clearance for marketing for the treatment of depression.

Jean-Pascal Lefaucheur and colleagues have examined repetitive Transcranial Magnetic Stimulation (rTMS) of the motor (pre-central) cortex for pain relief (Lefaucheur, J.-P., Drouot, X., Keravel, Y., and J.-P. Nguyen, “Pain relief induced by repetitive transcranial magnetic stimulation of precentral cortex,” Neuroreport: 17 Sep. 2001, 12:13, pp. 2963-2965, and Lefaucheur, J.-P., Hatem, S., Nineb, A., Ménard-Lefaucheur, I., Wendling, S., Keravel, Y., and J.-P. Nguyen, “Somatotopic organization of the analgesic effects of motor cortex rTMS in neuropathic pain,” Neurology 67:1998-2004, 2006). Lefaucheur (“Use of repetitive transcranial magnetic stimulation in pain relief,” Expert Review of Neurotherapeutics, May 2008, Vol. 8, No. 5, Pages 799-808, DOI 10.1586/14737175.8.5.799-(doi:10.1586/14737175.8.5.799) notes that a subset of patients will get relief from rTMS but relapse and for those patients surgically implanted epidural cortical electrodes and associated pulse generator can be proposed to allow pain relief more permanent, and that the rate of improvement due to rTMS may be predictive of the outcome of such an implantation.

In the medical literature, TMS coils are, by convention, almost always positioned with their handles pointing straight back, away from the face of the patient. This may also be referred to positioning along an anterior/posterior axis. In this position, the majority of the electric conventional current induced within the underlying brain will move from the back of the patient's head, toward the front of the patient's head, in line with the anterior/posterior (A/P) axis of the head. Standard coil positioning for the treatment of depression using TMS is accomplished in this manner in which induced current along the anterior-posterior axis predominates. Further, most conventional TMS reaches only the superficial cortical regions, and it is further uncertain how to orient one or more TMS coils when applying stimulation to deep brain targets.

In the article “Pain relief by rTMS: Differential effect of current flow but no specific action on pain subtypes” (Andre-Obadia N, Mertens P, Gueguen A, Peyron R, Garcia-Larrea L. Neurology 2008; 71:833-840), Andre-Obadia and colleagues test a “lateral-medial” (LM) coil position for a single coil over motor cortex region of the brain of pain patients. This “latero-medial position” is equivalent to the “transverse” positioning discussed herein. The authors conclude that this position is inferior to standard posterior-anterior (PA) positioning of the coil for producing analgesia, the latter of which produced a mean of 14% decrease in pain in the study (“PA positioning induces current predominantly along the anterior-posterior axis of the brain). In fact, Andre-Obadia and others asserted that not only did LM rTMS not outperform PA rTMS, it did not outperform placebo stimulation either. The Andre-Obadia article teaches against the application of TMS with magnet configuration oriented perpendicular to the posterior-anterior axis of the head. On the basis of the work of Andre-Obadia (such as that published in the article mentioned above) and others, those of skill in the art have been attempting TMS using standard PA positioning of the coils, and avoiding transferse positioning. Although posterior-anterior (PA) positioning of the coil is typically used, a few references have described the use of an approximately 45% to the long axis of the head. (Brasil-Neto 1992, Mills et al 1992). In addition, a few articles in the literature do suggest a role in activation of nerve fibers by electrical currents that pass those fibers in a transverse manner. A review and theoretical consideration for the merits of the case for transverse activation are described in Ruohonen et al 1996. Ruohonen however, is a theoretical paper that does not teach specific methods for achieving improved analgesia or antidepressant effects using transverse positioning, nor the targeted modulation of deep-brain white matter tracts in this manner.

Thus, it would be desirable to achieve improved levels of analgesia with a minimum of side-effects, using transcranial magnetic stimulation. It would also be desirable to achieve improved antidepressant and other clinical effects using transcranial magnetic stimulation.

### SUMMARY OF THE DISCLOSURE

Described herein are methods for modulating brain activity of one or more target brain regions, the methods using Transcranial Magnetic Stimulation (TMS) to modulate pain. For example, described herein are systems for arranging one or more (e.g. a plurality or array) of TMS electromagnet(s) in a predetermined position around a subject's head to provide significant analgesia. In particular, described herein are systems including a plurality individual TMS electromagnets that are positioned at predetermined locations around the patient's head, methods of positioning them, and using the positioned TMS electromagnets to create analgesia. The array may comprise different TMS electromagnets. For example, a top TMS electromagnet, a front TMS electromagnet, and two side (right side, left side) TMS electromagnets may be used. The TMS electromagnets may be bent figure-8 (e.g., two lobed) TMS electromagnets. In some variations, the principle direction of the current in each TMS electromagnet may be oriented with a specific polarity.

Surprisingly, when applying electromagnetic energy using an array as described herein, the arrangement of the TMS electromagnets, as well as their polarity, was found to have a profound effect on the amount of analgesia achieved, in some cases, positioning the TMS electromagnets in alternative positions and/or orientations actually resulted in an enhancement, rather than a diminishing, of the pain reported by the subject.

For example, described herein are methods of non-invasively treating pain by the application of Transcranial Magnetic Stimulation (TMS) using multiple TMS electromagnets to preferentially stimulate a patient's dorsal anterior cingulate gyrus relative to cortical brain regions. These method may include: positioning a top TMS electromagnet with an apex of the TMS electromagnet between about a Cz and Fz location on the patient's head; positioning a front TMS electromagnet with an apex of the TMS electromagnet between about an Fz and Fpz location on the patient's head; and modulating pain levels by applying stimulation from the top and front TMS electromagnets to the dorsal anterior cingulate gyrus, wherein the Cz, Fz and Fpz locations are determined using a standard 10-20 system for scalp electrode placement.

As used herein, the standard 10-20 system for scalp electrode placement typically refers to the 10-20 system (or International 10-20 system) that is an internationally recognized method to describe and apply the location of scalp electrodes in the context of an EEG test or experiment. This method is known by those of skill in the art and was developed to ensure standardized reproducibility between patients (e.g., subjects). This system is based on the relationship between the location of an electrode and the underlying area of cerebral cortex. The “10” and “20” refer to the fact that the actual distances between adjacent electrodes are either 10% or 20% of the total front-back or right-left distance of the skull. Each site has a letter to identify the lobe and a number to identify the hemisphere location. The letters F, T, C, P and O stand for frontal, temporal, central, parietal, and occipital lobes, respectively. Note that there exists no central lobe; the “C” letter is only used for identification purposes only. A “z” (zero) refers to an electrode placed on the midline. Even numbers (2, 4, 6, 8) refer to electrode positions on the right hemisphere, whereas odd numbers (1, 3, 5, 7) refer to those on the left hemisphere. In addition, the letter codes A, Pg and Fp identify the earlobes, nasopharyngeal and frontal polar sites respectively.

Two anatomical landmarks are used for the essential positioning of the EEG electrodes: first, the nasion which is the distinctly depressed area between the eyes, just above the bridge of the nose; second, the inion, which is the lowest point of the skull from the back of the head and is normally indicated by a prominent bump. In some variations, extra positions are added using the 10% division, which fills in intermediate sites halfway between those of the standard 10-20 system. This modified (though still “standard”) position naming-system is referred to as the Modified Combinatorial Nomenclature (MCN). This MCN system uses 1, 3, 5, 7, 9 for the left hemisphere which represents 10%, 20%, 30%, 40%, 50% of the inion-to-nasion distance respectively. The introduction of extra letter codes allows the naming of intermediate sites. Note that these additional letter codes do not necessarily refer to an area on the underlying cerebral cortex. The additional letter codes for intermediate sites are: AF—intermediate between Fp and F, FC—between F and C, FT—between F and T, CP—between C and P, TP—between T and P, PO—between P and O. Also, the MCN system renames four points of the 10-20 system—T3, T4, T5 and T6—asT7, T8, P7 and P8 respectively.

As used herein, the 10-20 (and any derivative) naming system refers to the position relative to the patient's head, and does not require electrode placement, but is instead adapted for positioning of the TMS electromagnets (e.g., the apex of a TMS electromagnet). The “apex” of a TMS electromagnet is typically the region, which may be, but does is not necessarily, the center of the most distal portion of the TMS electromagnet intended to be positioned near and/or against the subject's head. The apex may be the region of highest emitted field from the TMS electromagnet.

In some variations, positioning the side TMS electromagnets may also include positioning an apex of a side TMS electromagnet on the right side of the patient's head. For example, the right side TMS electromagnet may be placed on the right side of the patient's head between C4 and Fp2, e.g., between C4 and F4, between Fp2 and F4, or more anteriorly, e.g., between F8 and Fp2 on the patient's head. These locations are determined using a standard 10-20 system for scalp electrode placement. In some variations, positioning side TMS electromagnets may include positioning an apex of a left side TMS electromagnet on the left side of a patient's head. For example, the left side TMS electromagnet may be placed on the left side of the patient's head between C3 and Fp1, e.g., between C3 and F3, between F3 and Cp1, or more anteriorly, e.g., between F7 and Fp1 on the patient's head. As mentioned, the locations are determined using a standard 10-20 system for scalp electrode placement.

In some variations, the top TMS electromagnet is positioned within about 2.5 cm anterior of Cz (e.g., within about 2 cm anterior of Cz, etc.). The front TMS electromagnet may be positioned within about 2 cm anterior to Fz (e.g., within about 1 cm anterior of Fz).

In general, any appropriate TMS electromagnet may be used, including “figure-8” type TMS electromagnets. However, many such TMS electromagnets may be large and therefore difficult to position within the constraints of the methods described. Thus, in some variations it may be advantageous to use one or more TMS electromagnets having a bent (e.g., V-shaped, swept-wing, etc.) configuration so that the apex of the TMS electromagnet is narrower than the more distal regions, allowing it to be positioned close to the patient's skull and near other TMS electromagnets. For example, in some variations, the front and top TMS electromagnet comprise bent figure-8 TMS electromagnets. For example, the top TMS electromagnet may comprise a swept-wing TMS coil.

In some variations, the rate of stimulation may be controlled to modulate pain. For example, the modulation of pain levels by applying stimulation from the top and front TMS electromagnets to the dorsal anterior cingulate gyrus may comprise applying stimulation at a frequency of stimulation from the front and top TMS electromagnets that is above about 1 Hz, above about 2 Hz, above about 5 Hz, above about 7 Hz, above about 10 Hz, etc.

In general, modulation of pain may include inducing analgesia or, in some variations, enhancing pain. For example, modulation of pain level may comprise reducing pain by applying stimulation from the top and front TMS electromagnets to the dorsal anterior cingulate gyms. Any type of pain may be modulated, and in particular chronic pain. For example, the methods described herein may be applied to modulate (e.g., reduce) the pain of fibromyalgia.

In some variations, described herein are methods of non-invasively treating pain by the application of Transcranial Magnetic Stimulation (TMS) using multiple TMS electromagnets to preferentially stimulate a patient's dorsal anterior cingulate gyrus relative to cortical brain regions, the method comprising: positioning a top TMS coil anterior to a Cz location on the patient's head so that the principle direction of electrical current induced by the electromagnet is in the anterior-posterior axis of the patient's head; positioning a front TMS coil anterior to an Fz location on the patient's head; positioning a side coil on the left side of the patient's head, and; positioning a side coil on the right side of the patient's head; and reducing pain levels by applying stimulation from the TMS electromagnets to the dorsal anterior cingulate gyrus, wherein the Cz, Fz, C3, C4, F3 and F4 locations are determined using a standard 10-20 system for scalp electrode placement.

In general, the methods described herein may be performed with device and systems specifically adapted to apply TMS as indicate by the methods. For example, an applicator may be configured to hold the TMS electromagnets in the predetermined appropriate configurations described herein. In variations having an array of four TMS electromagnets, the applicator may be configured to be positioned around a patient's head so that the rough positions of the TMS electromagnets are approximately over the appropriate region of the patient's head (e.g., at, between or near the Cz, Fz, C3, C4, F3 and F4 locations). An applicator may be configured to connect with a TMS system including power sources to drive TMS. The applicator may include a frame or framework holding a plurality (e.g., two, three, four, or more) mounts that adjustably hold the TMS electromagnets. The gross relative positions of the mounts in the applicator may be fixed in the engaged configuration, when the applicator is positioned over the patient's head. However, the fine positions of the mounts may be adjustable to more precisely position the TMS electromagnets. In some variations the relative position of the mounts are fixed in a predetermined position when the applicator is in the engaged (e.g., TMS delivery) configuration. The applicator may also include a second configuration for inserting or removing the patient's head. For example, the applicator may be configured with a movable (e.g., hinged, pivoting, etc.) arm that moves one or more of the applicators (and therefore any TMS electromagnet) away from the engaged configuration to “open up” the applicator so that the patient's head can be inserted or withdrawn from the applicator.

In general, the mounts for holding the TMS electromagnets may be configured to adjustably hold the TMS electromagnets. Each mount may be configured so that the TMS electromagnet can be moved radially (e.g. inwards, towards the head of the patient, or outwards, away from the head of the patient) when the applicator is around a patient's head. The mount may also be configured to allow the TMS electromagnet to adjust the angle of the TMS electromagnet relative to the framework of the applicator (and/or relative to the patient's head). Each mount may also be configured to adjust the fine position of the TMS electromagnet held by the mount. The mount may allow rotation of the TMS electromagnet, thereby allowing adjustment of the polarity of the TMS electromagnet. However, in some variations, one or more of the mounts is configured to hold a TMS electromagnet in one or more predetermined positions providing known polarities relative to the patient. In some variations, the mount is configured to allow the TMS electromagnet to be pivoted about the apex of the TMS electromagnet (e.g., the contact point with the patient).

For example, described herein are Transcranial Magnetic Stimulation multi-electromagnet applicators configured to be positioned over a patient's head for non-invasively treating pain by the application of Transcranial Magnetic Stimulation (TMS) using multiple TMS electromagnets to preferentially stimulate a patient's dorsal anterior cingulate gyms relative to cortical brain regions. An applicator may include: a framework comprising a first mount, a second mount, a left side mount, and a right side mount, wherein the framework holds a plurality of TMS electromagnets in a predetermined arrangement around the patient's head so that when the framework is positioned over the patient's head, a first TMS electromagnet is between about a Cz and Fz location on the patient's head, a second TMS electromagnet is between about an Fz and Fpz location on the patient's head, a left side TMS electromagnets is on the left side of the patient's head, and a right side TMS electromagnet is on the right side of the patient's head; and the first mount, second mount, left side mount, and right side mount are each configured to secure a TMS electromagnet to the framework and are further configured to allow adjustment of the angle of the TMS electromagnet relative to the framework, and to allow adjustment of a radial distance of the TMS electromagnet from the frame and toward the surface of the patient's head when the framework is positioned over the patient's head, wherein the Cz, Fz and Fpz locations are determined using a standard 10-20 system for scalp electrode placement.

As mentioned, in some variations the framework comprises a movable (e.g., hinged) region configured to move one or more of the first mount, second mount, left side mount, and right side mount out of the predetermined arrangement so that the device can be positioned over the patient's head.

As mentioned, the first mount, second mount, left side mount, and right side mount may each be configured to hold a TMS electromagnet so that the TMS electromagnet is pivotable about a contact point with the patient's head when the framework is positioned over the patient's head. For example, the first mount, second mount, left side mount, and right side mount may each comprise a ball joint.

In some variations, the applicator (or a system including the applicator) includes one or more (or all) of the TMS electromagnets. For example, the applicator may include a top TMS electromagnet within the first mount, a front TMS electromagnet within the second mount, a left side TMS electromagnet within the left side mount, and a right side TMS electromagnet within the right side mount. As mentioned, any appropriate TMS electromagnet may be used. For example, the top TMS electromagnet, front TMS electromagnet, left side TMS electromagnet, and right side TMS electromagnet may all be bent TMS coils.

In some variations, the framework may be configured so that when the framework is positioned over the patient's head, the left side TMS electromagnet is between the C3 and F3 locations, wherein the C3 and F3 locations are determined using a standard 10-20 system for scalp electrode placement. Similarly, the framework may be configured so that when the framework is positioned over the patient's head, the right side TMS electromagnet is between the C4 and F4 locations, wherein the C4 and F4 locations are determined using a standard 10-20 system for scalp electrode placement.

In some variations, a Transcranial Magnetic Stimulation multi-electromagnet applicator configured to be positioned over a patient's head for non-invasively treating pain by the application of Transcranial Magnetic Stimulation (TMS) using multiple TMS electromagnets to preferentially stimulate a patient's dorsal anterior cingulate gyrus relative to cortical brain regions includes: a top mount configured to hold a top TMS electromagnet; a front mount configured to hold a front TMS electromagnet; a left side mount configured to hold a left side TMS electromagnet; a right side mount configured to hold a right side TMS electromagnet; and a framework holding the top mount, front mount, left side mount, and right side mount in a predetermined configuration so that when the device is positioned over the patient's head, the top TMS electromagnet is positioned between about a Cz and Fz location on the patient's head, the front TMS electromagnet is between about an Fz and Fpz location on the patient's head, the left side TMS electromagnets is on the left side of the patient's head, and the right side TMS electromagnet is on the right side of the patient's head; wherein each mount of the top mount, front mount, left side mount and right side mount allow adjustment of the angle and radial distance of a TMS electromagnet held by each mount relative to the framework, wherein the Cz, Fz and Fpz locations are determined using a standard 10-20 system for scalp electrode placement.

In general, as will be shown by the figures and described in the text below, the devices and methods descried herein result in deep-brain stimulation of the patient. The net effect is a statistically significant effect (e.g., analgesia) compared to other TMS electromagnet orientations, which may not have a statistically significant effect. The methods and systems described herein are primarily multi-coil TMS systems. The orientation of the coils may be used to determine the aggregate effect and orientation on the deep brain target.

Applying either low-frequency (e.g., less than about 5 Hz, less than about 2 Hz, etc.) or high frequency stimulation (5 Hz or greater, for example 10 Hz) produces significant analgesia acutely. In some cases, high frequency stimulation produces significant pain reduction in patients with a chronic pain condition.

In general, the methods of treatment described herein including methods of treating a patient by applying Transcranial Magnetic Stimulation (TMS). The method may include the steps of: positioning a plurality of TMS electromagnets outside of a subject's head towards a target brain region; and treating the patient by applying stimulation from the TMS electromagnet to the target brain region. The treatment may generally be directed to a therapeutic treatment such as the treatment of depression, the relief of pain, etc. The target may be a deep brain target, and may be a target associated with a desired therapeutic effect.

For example, described herein are methods of modulating brain targets such as the Dorsal Anterior Cingulate Gyms (DACG) so as to produce robust analgesia by the application of low-frequency Transcranial Magnetic Stimulation (TMS) that include the steps of positioning a TMS electromagnet outside of a subject's head towards a target brain region so that the principle net direction of current flowing in the TMS electromagnet is transverse to the A-P axis of the subject's head; and evoking significant analgesia by impacting the target brain region(s) by applying a low-frequency stimulation from the TMS electromagnet.

The step of modulating brain activity may include applying a frequency below 5 Hz, or below 2 Hz (e.g., between about 0.5 and 2 Hz). The step of modulating brain activity may include applying a frequency above 5 Hz. Such high frequency TMS (for example trains of 10 Hz pulses) may be of particular utility in treating chronic pain conditions, as well as depression.

In some variations, the step of positioning comprises positioning a plurality of TMS electromagnets outside of the subject's head towards the target brain region so that the principle direction of current in at least one of the TMS electromagnets is transverse to the A-P axis of the subject's head.

Positioning an array of TMS electromagnets may comprise positioning a plurality of electromagnets outside of the subject's head towards the target brain region. As mentioned above, an applicator, which may include a frame (e.g., gantry, clamp, arm, helmet, or other “holder”) may be used to hold the plurality of TMS electromagnets in position relative to the patient's head. The frame may be configured to allow adjustment (to each patient or to different target deep brain regions) of one or more of the TMS electromagnets, and may be further configured to lock or hold them in place for or during the application of energy.

Any appropriate target brain region may be chosen, particularly deep brain regions. For example, the target brain region may be the Dorsal Anterior Cingulate Gyrus. For example, positioning a TMS electromagnet may comprise positioning the TMS electromagnet so that the principle direction of current in the electromagnets is transverse to the cingulate gyrus.

Another deep brain target region includes the medial forebrain bundle. The method may include positioning the TMS electromagnet by positioning the TMS electromagnet so that the principle direction of current in the electromagnets is transverse to the medial forebrain bundle. In some variations, however, it has been found that the principle direction of current in one or more of the electromagnets is not transverse, but parallel to the anterior-posterior axis.

Also described herein are methods of reducing pain by the application of Transcranial Magnetic Stimulation (TMS) using an array of TMS electromagnets arranged to target the dorsal anterior cingulate gyrus.

### BRIEF DESCRIPTION OF THE DRAWINGS

FIG. 1 illustrates conventional TMS coil positioned relative to a brain using a single FIGURE-8 TMS coil oriented so it is parallel to the anterior-posterior axis of the head.

FIG. 2 illustrates TMS using an array of TMS coils arranged with the main axis of current along an anterior/posterior line.

FIG. 3 is a bar graph showing the analgesia effect resulting from employing the TMS configuration shown in FIG. 2.

FIG. 4 illustrates a traditional figure-8 TMS coil oriented so that it is transverse to the anterior-posterior axis of the head.

FIG. 5 illustrates transverse activation using an array of four double-coil TMS electromagnets.

FIG. 6 demonstrates robust analgesia resulting from employing the TMS configuration shown in FIG. 5.

FIG. 7 illustrates the location of the patient-contacting surfaces of the coils in one variation of a 4-coil transverse array, as related to a figure of a human head.

FIG. 8 shows the positions for each of the coils in the 4-coil transverse array positions on the head with respect to standard EEG-10-20 scalp locations (in the “B” configuration).

FIG. 9 illustrates the location of the patient-contacting surfaces of the coils within a 3-coil transverse array, as related to a figure of a human head.

FIG. 10 shows the positions for each of the coils in the 3-coil transverse array positions on the head with respect to standard EEG-10-20 scalp locations.

FIG. 11 documents actual pain reduction results in patients with fibromyalgia comparing different array orientations.

FIG. 12 shows the acute pain reduction effect of a transverse 4-coil array when operated at 10 Hz, 3000 pulses in the orientation indicated herein.

FIG. 13 documents the antidepressant effect of the 4-coil transverse array operated at 10 Hz as compared with 45-degree multi-coil array.

FIG. 14 shows a table of magnetic field vectors associated with transverse coil arrays as compared with a standard single coil in standard (A/P-oriented) positioning. Values represent B-field power produced by 1 amp DC simulation input to each coil, and are directly proportional to the values that are produced when powering each coil with an actual TMS pulse

FIG. 15 identifies the axes referred to in FIG. 14 with respect to a human head.

FIG. 16A-C illustrate the positions and principle direction of electrical current induced by the electromagnet for one configuration (the “A” configuration) of a four-TMS electromagnet array.

FIGS. 17A-C illustrate the positions and principle direction of electrical current induced by the electromagnet for one configuration (the “B” configuration) of a four-TMS electromagnet array.

FIGS. 18A-C illustrate the positions and principle direction of electrical current induced by the electromagnet for one configuration (the “C” configuration) of a four-TMS electromagnet array.

FIGS. 19A-C illustrate the positions and principle direction of electrical current induced by the electromagnet for one configuration (the “D” configuration) of a three-TMS electromagnet array.

FIG. 20 shows one variation of a four-TMS electromagnet array in an applicator.

FIGS. 21A and 21B shows side and front perspective views, respectively, of a human skull to illustrate methods of orienting and placing TMS electromagnets relative to a patient's head.

FIG. 22 illustrates the ratio of dorsal Anterior Cingulate Gyms (dACG) versus surface activity modulation from PET data for each of four configurations (configurations A-D) of exemplary systems as described herein.

FIG. 23 compares the various configurations of TMS electromagnetic systems by finite element analysis (FEA) of the various TMS stimulation components in the dorsal Anterior Cingulate Gyms.

FIG. 24 illustrates the relationship between genual dorsal Anterior Cingulate Gyrus modulation and analgesia.

FIG. 25 illustrates the effects of different configurations and stimulation parameters for TMS systems in patients with Fibromyalgia over time.

### DETAILED DESCRIPTION OF THE INVENTION

In general, described herein are TMS treatment systems, devices and methods for neuromodulation. In particular, the systems and methods described herein may be used to help treat pain (e.g., for analgesia). These systems and methods may be particularly configured for the neuromodulation of both superficial and/or deep-brain targets, including the dorsolateral prefrontal cortex, Dorsal Anterior Cingulate Gyms (cingulate gyms), medial forebrain bundle, etc.

Although the inventors do not wish to be bound by any particular theory of operation, one mechanism by which the transverse arrays described herein might exert their effect is direct action of magnetic fields upon deep white matter tracts (such as the cingulate bundles or medial forebrain bundle) in response to transverse induced electrical currents. Such types of neuromodulation are described in greater detail for example, in U.S. patent application Ser. No. 12/324,227, titled “TRANSCRANIAL MAGNETIC STIMULATION OF DEEP BRAIN TARGETS,” now U.S. Pat. No. 8,267,850, which is incorporated by reference in its entirety.

As used herein, the term “deep brain” refers to the region of the patient's brain that deeper within the brain than the outer cortical regions of the brain. Although the outer cortical regions maybe stimulated using the methods, devices and systems described herein, the deep brain regions are of particular interest. Examples of deep brain regions may include (but are not limited to): subthalamic nucleus, globus pallidus extema, anterior cingulate gyrus, posterior cingulate gyms, subgenual cingulate gyrus, anterior cingulate, dorsal cingulate gyrus, ventromedial nucleus of thalamus, ventrolateral nucleus of thalamus, anterior limb of the internal capsule, nucleus accumbens, septal nucleus, hippocampus, medial forebrain bundle, etc.

Although the examples and illustrations described herein typically include a plurality of TMS electromagnets (TMS coils), one, two, three, four or more TMS electromagnet coils may be used. The system may generally include a frame/framework (e.g., a scaffold, holder, arm, gantry, or the like) to hold the one or more TMS electromagnets in position outside of a patient's head for TMS application. The frame may be adjustable; in some variations the frame includes preset locking positions for holding the TMS electromagnets in position target the brain regions.

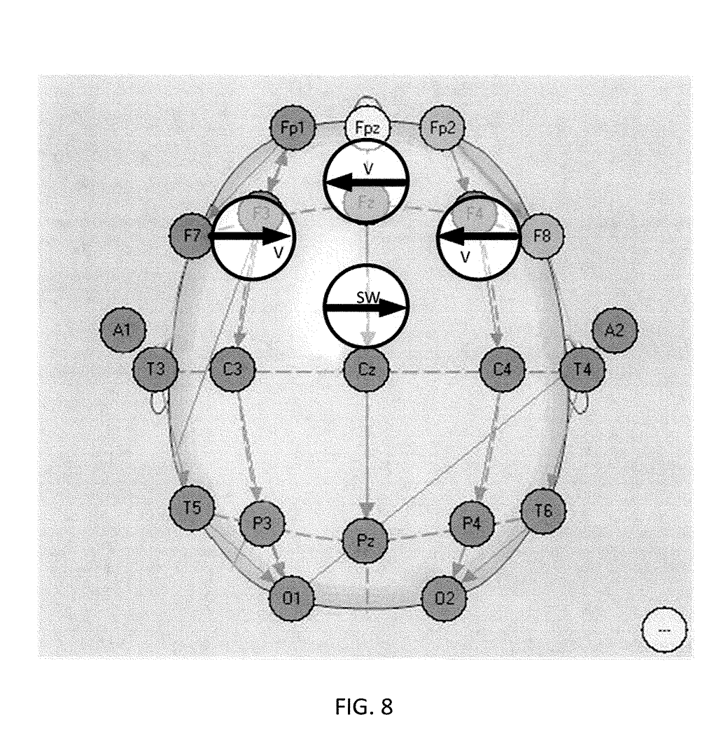

Any appropriate TMS electromagnet(s) may be used, including traditional “FIG. 8” TMS coils, as well as bent TMS coils, such as swept-wing TMS coils, V-shaped TMS coils, and the like. A bent and/or swept-wing TMS coil may include a plurality of coil windings that meet at a central region and extend outward from the central region (which may be “flat” in swept-wing embodiments) out of the plane of the central region of the magnet. Examples of such TMS coils are illustrated in International Application No. PCT/US2010/020324, titled “SHAPED COILS FOR TRANSCRANIAL MAGNETIC STIMULATION,” filed on Jan. 7, 2010, Publication No. WO 2010/080879, previously incorporated by reference in its entirety.

For example, described herein are systems and methods for modulating brain regions, including deep brain target regions, using one or more TMS electromagnets configured for stimulation (including low frequency stimulation) to achieve robust anesthesia by neuromodulating target brain regions. Some of the targets for treatment of pain are the Dorsal Anterior Cingulate Gyms (DACG), the prefrontal cortex, and the motor cortex. Neuromodulation producing analgesia of targets such at the DACG may involve down-regulation at that target. In particular, as described herein, robust anesthesia caused by neuromodulation of target brain regions may be achieved using low or high-frequency TMS. In some variations, the primary direction of current in the TMS coil is perpendicular to the anterior-posterior axis of the head (e.g., the AP axis of the head, skull, brain, etc.). This configuration is called the transverse configuration. The primary direction of current may be the result the combined (e.g., summed) effect of a plurality of TMS electromagnets; thus, it may not be an individual direction or polarities of a particular TMS electromagnet in an array that is relevant but instead the combined effect of these multiple TMS electromagnets.

In some variations, the methods of performing TMS to treat a patient include treatment at low frequency to neuromodulate the target brain regions by orienting the TMS coil so that the direction of the primary current in the TMS coil is transverse to the Anterior-Posterior axis of the head. (The direction of the electrical current induced by the TMS is typically opposite to the direction of the primary applied current within the coil windings, thus along the same axis). Thus, in the invention herein described, the direction of the induced electrical current within the brain is perpendicular to the AP axis of the head.

Low frequency stimulation may include stimulation at or below 5 Hz (e.g., 5 Hz, 1 Hz, etc.). In some variations the frequency may be greater than 5 Hz, 10 Hz, etc. (e.g., between 5 Hz and 20 Hz, 10 Hz and 20 Hz, between 10 Hz and 50 Hz, etc.), which is termed herein “high-frequency” stimulation.

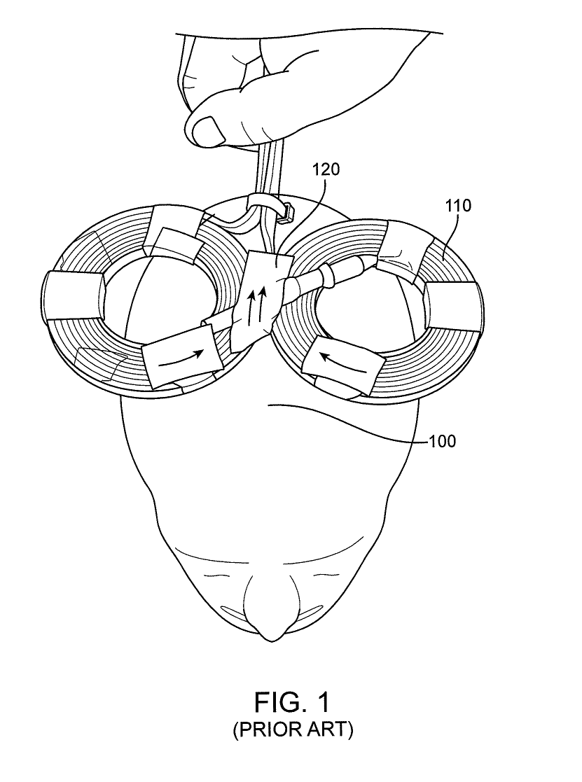

FIG. 1 illustrates a conventional TMS coil positioned relative to a brain using a single FIGURE-8 TMS coil oriented so it is parallel to the anterior-posterior axis of the head. The main direction of the induced electrical currents (e.g., near the center region of the TMS coil) are in the direction of the A-P axis of the head.

In FIG. 1, patient-head representation 100 is shown with figure-8 double-coil 110 (without its shield so that actual coils can be viewed) with arrows 120 illustrating the current flow where both coils have, in the double-coil center, the current flowing in the same direction. This example illustrates the standard and accepted method of positioning a TMS electromagnet. The electrical current induced in the target will be in opposite direction to arrows 120, along the A/P axis of the head. This is the traditional orientation in which TMS coils are used over patient's heads, and is used as a figure in order to illustrate the prior art.

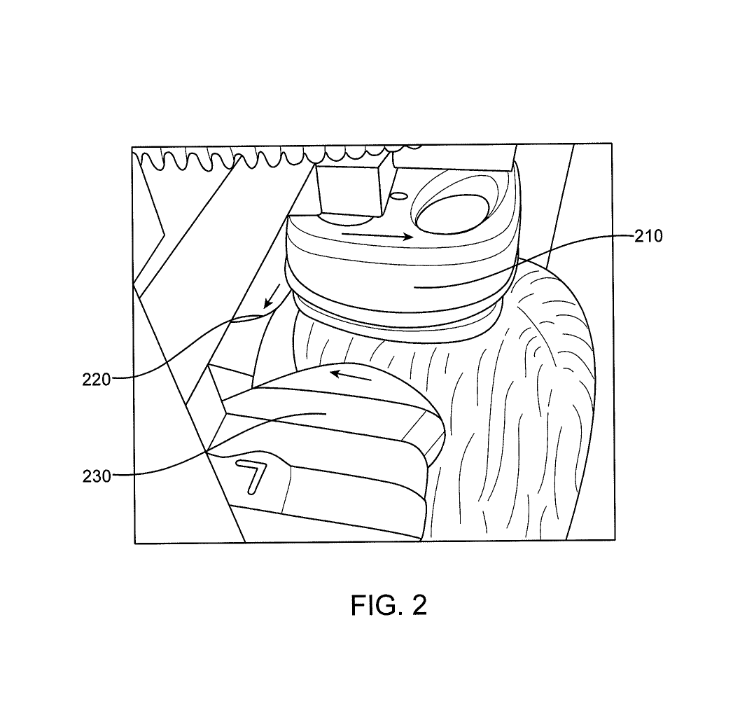

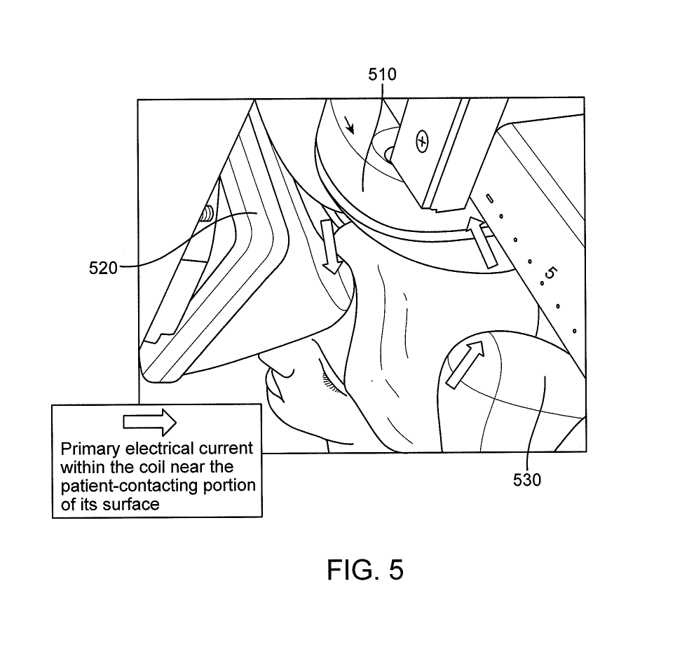

FIG. 2 shows an array of four TMS electromagnets oriented approximately along the A-P axis of the head. In FIG. 2, the primary directions of current (e.g., near the center regions of the TMS coils) are in the direction of the A-P axis of the head. The array of TMS electromagnets in FIG. 2 shows four double-coil TMS electromagnets of different configurations, including V-shaped or swept-wing coils. The four electromagnet coils are the swept-wing top double-coil 210, V-double-coil front coil 220, and V-double-coil side coil 230. Opposite V-double-coil side coil 230 is a companion V-double-coil side coil. In FIG. 2, the arrows shown on the TMS electromagnets indicate the primary direction of electrical current in each double coil structure at their geometric centers. In some examples, when the TMS magnets are oriented with the main axis of current (shown by arrows on each TMS coil in FIG. 2) oriented along an anterior/posterior line, stimulation (e.g., 1 Hz) results in down-regulation of affected brain tissue. As described in greater detail below, this A/P-oriented configuration was found to be surprisingly less effective for achieving analgesia when used to stimulate particular deep-brain target regions. Instead, configurations in which one or more of the TMS electromagnets (or the net effect of the TMS electromagnets) results in transverse current at the deep brain target region, e.g., transverse to the A-P axis showed superior performance in inducing analgesia, and better ability to reach the deep dorsal anterior cingulate region in brain imaging studies. For example, the orientation described below for FIG. 5 was surprisingly superior in performance compared to the arrangement illustrated in FIG. 2.

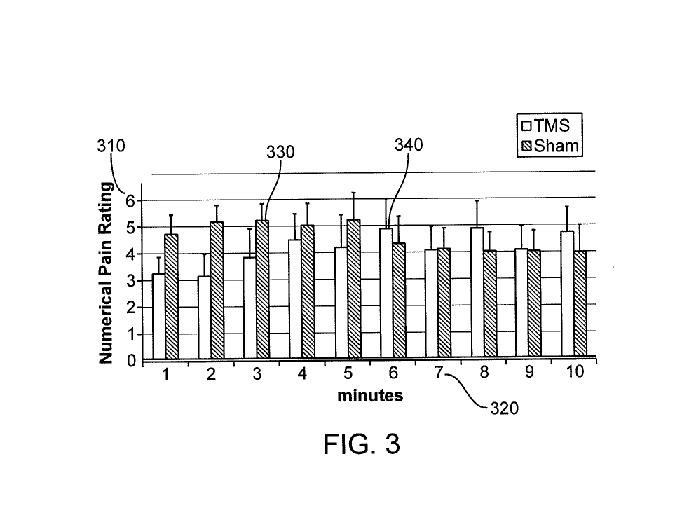

FIG. 3 illustrates the clinical analgesic effect of slow-rate stimulation in the configuration shown in FIG. 2 on various target structures affected by the TMS after a pain stimulus was applied. To do the pain study, the skin of the subject is first sensitized with capsaicin. This is followed by the heat threshold and tolerance being determined with Peltier thermode over the sensitized area. Then pain a stimulus is administered with a Peltier thermode at constant temperature over sensitized area where the stimulus corresponds with 60% tolerance level. Verbal reports on pain level were obtained every one minute for ten minutes. In this example, a subject was either stimulated using low-frequency stimulation or was sham stimulated. For an average of seven subjects, the Numerical Pain Rating on a scale of 2 to 10 as judged by the subject is shown in FIG. 3 with pain-ranking axis 310 versus points on minutes-of-pain-stimulus scale 320 with bars 330 representing the average pain score for the sham condition and bars 340 representing the average pain score for the TMS-stimulation conditions. The overall average was an approximately 30% reduction in reported pain. Overall, the activity in the target brain regions (e.g., DACG, which usually becomes more active in the presence of pain) was lowered following real versus sham magnetic stimulation, indicating a down-regulation in activity, and this down-regulation was accompanied by significant analgesia.

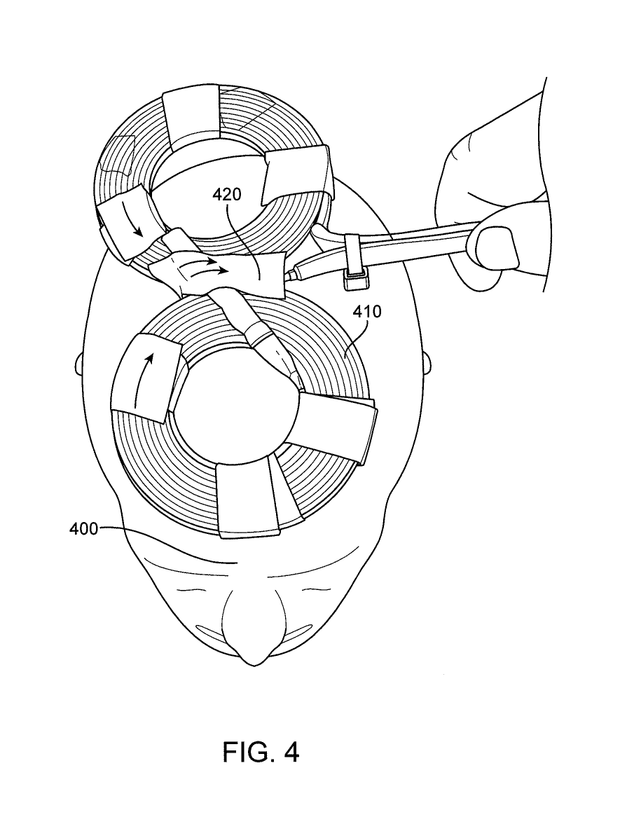

In contrast, the present invention achieved substantially more clinical analgesia when the orientation of the TMS electromagnet(s) is/are rotated by approximately 90 degrees, so that the principle direction of the current in the TMS electromagnet(s) is oriented transverse to the A-P axis of the head. FIG. 4 illustrates a traditional figure-8 TMS coil oriented so that it is transverse to the anterior-posterior axis of the head. The primary direction of the currents (e.g., near the center region of the TMS coil, is transverse to the direction of the A-P axis of the head. In FIG. 4, patient-head representation 400 is shown with figure-8 double-coil 410 (without its shield so that actual coils can be viewed) with arrows 420 illustrating the current flow where both coils have, in the double-coil center, the current flowing in the same direction. The electrical current induced in the target will be in opposite direction to arrows 420.

In FIG. 5 transverse activation is illustrated using a four double-coil array. The four electromagnet coils are the swept-wing top double-coil 510, V-double-coil front coil 520, and V-double-coil side coil 530. Opposite V-double-coil side coil 530 is a companion V-double-coil side coil. Arrows indicate the main direction of primary electrical current in each double coil structure at their geometric centers. Slow-rate stimulation while in this novel orientation (e.g., at 1 Hz) results in robust analgesia due to neuromodulation of affected brain tissue, when compared to the analgesia obtained with the multiple-coil array in the AP configuration.

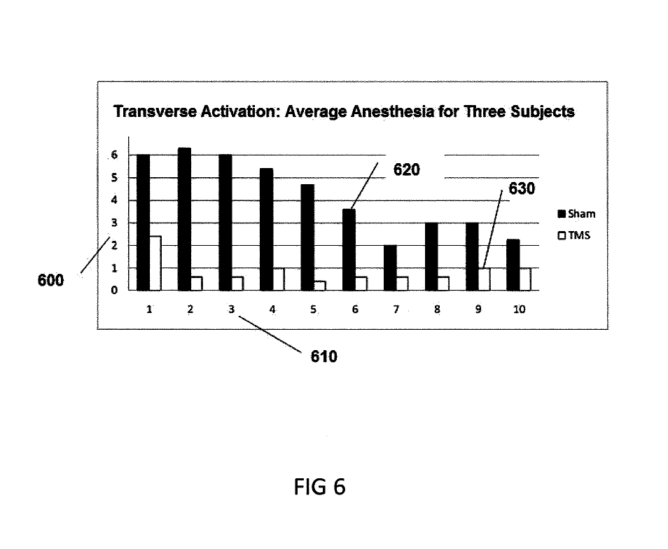

FIG. 6 illustrates the 60 to 94% reduction in pain obtained in studies of three subjects using the transverse orientation. In FIG. 6, Numerical Pain Score Rating scale 600 is used to evaluate pain levels 620 for Sham stimulation and 630 for TMS stimulation where the pain measurements were taken minute-by-minute over a ten-min period with the minutes marked in 610. The pain reduction in this transverse case is significantly greater than the 30% reduction shown in FIG. 3 where the coil array was oriented parallel to the anterior-posterior axis of the head.

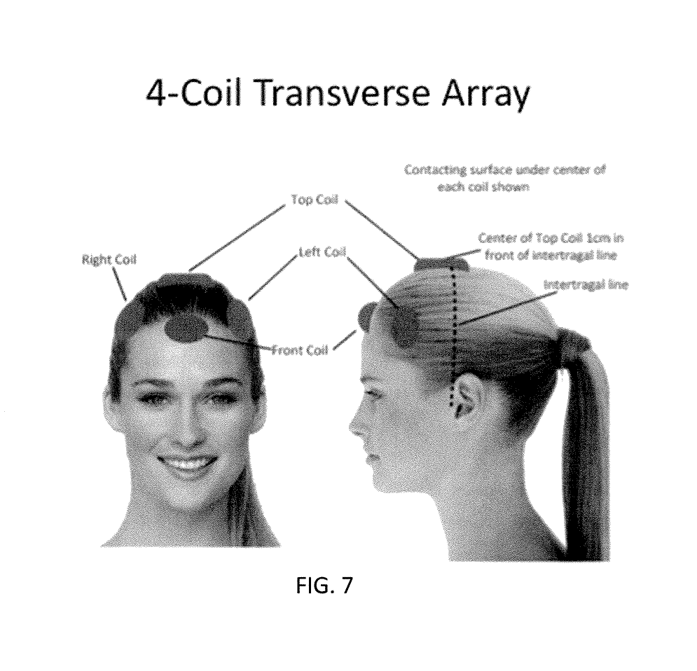

The arrangements of TMS coils used to achieve the results illustrated in FIG. 6 are shown below. In this example, an array of TMS coils of various types (swept-arm and V-shaped TMS coils) are arranged around the patient's head and held in place using a frame that holds the coils so that the primary induced current from one or all of the TMS coils will be oriented (as confirmed by simulation of the applied fields) transverse to the A-P axis of the patient's head. For example, FIG. 7 illustrates the locations of the patient-contacting surfaces of four coils within a 4-coil transverse array, as related to a figure of a human head. The shortest line between the ears on each side of the head may be used as a guide to center the top coil forward of that line so as to avoid inadvertent motor cortex stimulation. Placement of the TMS coils using this arrangement generally may be modified depending on the particular deep-brain target region. For example, the deep-brain target region may be oriented for transverse deep-brain stimulation of the deep-brain target.

FIG. 8 shows the positions for each of the coils in the 4-coil transverse array positions on the head (as shown in FIG. 7) with respect to standard EEG-10-20 scalp locations. The large circles represent each of the four coils in the array. For example, the top coil may be of a swept-wing (“SW”) design, and be located anterior to Cz, anterior and clear of motor cortex. Left and right side coils may be of V-coil design (“V”) and be placed approximately at F3 and F4, respectively. A front coil may also be of the V design and be placed approximately over Fz. The arrow within each coil representation circle may indicate the direction of the primary electrical current within the coil near the patient-contacting portion of its surface. The effect of such a coil is generally to drive induced electrical current within the brain in a direction opposite of primary electrical current. In this transverse 4-coil array, the primary current within the coil at the patient-contacting (center) of the top coil is directed to the patient's right; the front coil to the patient's left, and the left and right side coils, upward. In FIG. 8, the large circles represent scalp-contacting surface of coil centers, as mentioned, arrows in the circles represent the direction of primary electrical current within the coil near the patient contacting portion of its surface

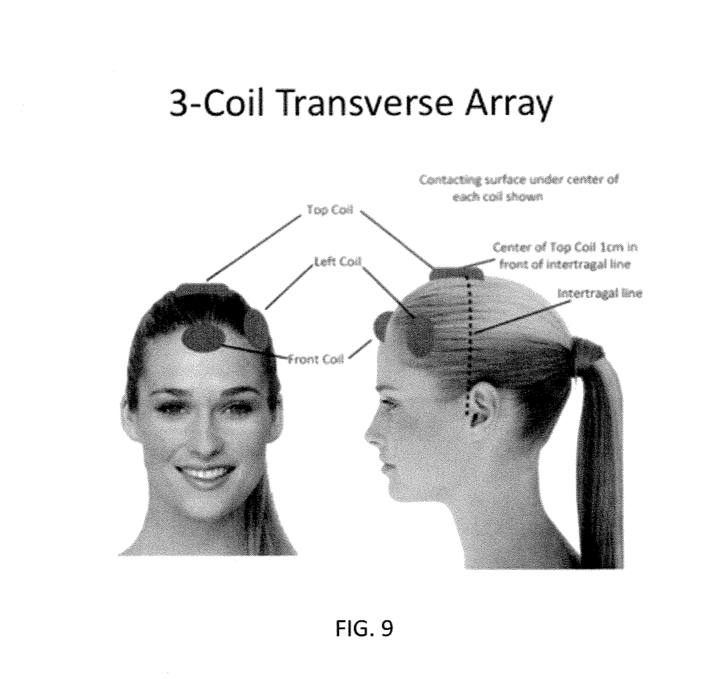

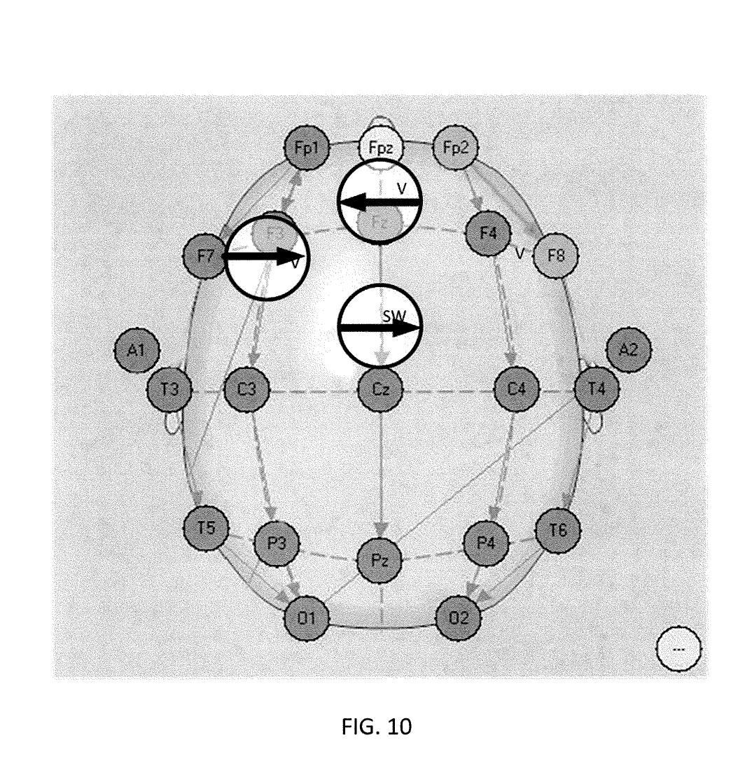

Another alternative is shown in FIG. 9. FIG. 9 illustrates the location of the patient-contacting surfaces of the coils within a 3-coil transverse array, as related to a figure of a human head. The shortest line between the ears on each side of the head may be used as a guide to center the top coil forward of that line so as to avoid inadvertent motor cortex stimulation. FIG. 10 shows the positions for each of the coils in the 3-coil transverse array positions of FIG. 9 on a head with respect to standard EEG-10-20 scalp locations. The large circles represent each of the four coils in the array. In this example, the top coil may be of a swept-wing (“SW”) design, and be located anterior to Cz, anterior and clear of motor cortex. Left side coil (or alternatively a right side coil) may be of V-coil design (“V”) and be placed approximately at F3. The front coil may also be of the V design and be placed approximately over Fz. The arrow within each coil representation circle indicates the direction of the primary electrical current within the coil near the patient-contacting portion of its surface. The effect of such a coil is generally to drive induced electrical current within the brain in a direction opposite of primary electrical current. In this transverse 3-coil array, the primary current within the coil at the patient contacting (center) of the top coil is directed to the patient's right; the front coil to the patient's left, and the left side coil is upward.

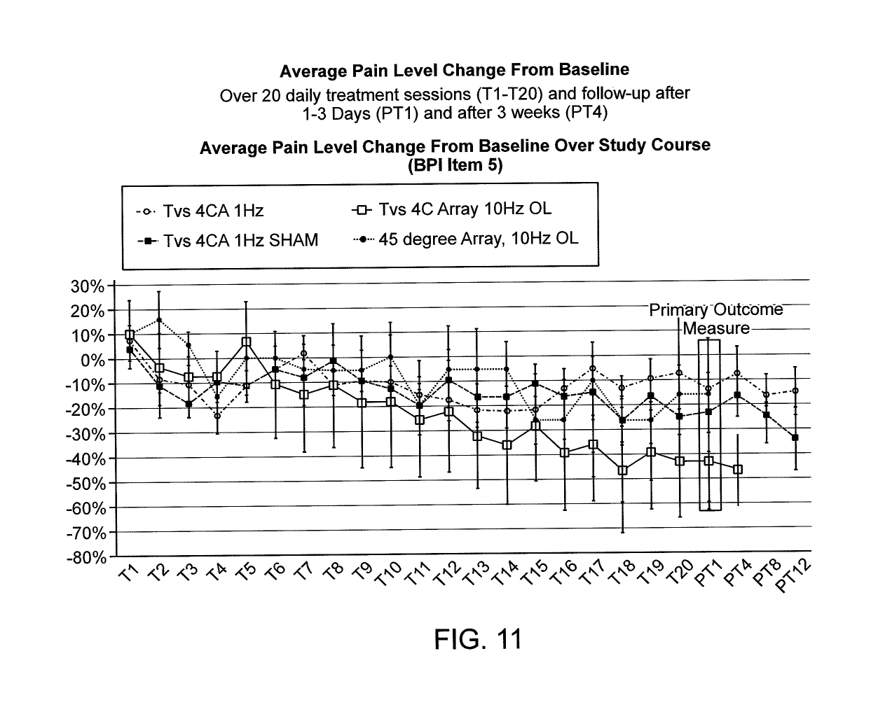

FIG. 11 documents actual pain reduction results in patients with fibromyalgia, a chronic disease condition in which pain is a significant feature. “Average pain level over the last 24 hours” is reflected in the score of Item 5 in the standard Brief Pain Inventory. Over a series of 20 TMS treatment sessions and follow-up period, the scores from each of four different treatment groups are shown as averages for that group. The first group is shown with small dashed lines, and refers to patients for whom receiving traditional stimulation (not transverse to the AP axis) at 1 Hz. The second group, with alternating short dashed lines and dots is from patients receiving sham stimulation (at 1 Hz). The third group, represented by a thick black line and heavy square boxes, represents treatment with a transverse 4-coil array energized with 10 Hz pulse trains using the same traditional orientation of the array of TMS electromagnets. Finally, the fourth group, represent by a line consisting of dots only is an open-label test of a multi-coil array in which coils are turned to a 45-degree angle with respect to the anterior-posterior axis of the head, and energized with 10 Hz pulse trains. While all groups show reduction in pain levels relative to baseline (including the sham), only the line for the transverse 4-coil array operated at 10 Hz surpasses 30% pain reduction line (an industry standard for clinical utility), and reaches an average 42% reduction at the time of the pre-designated primary outcome measure. As shown in FIG. 11, this improvement is maintained and possibly improved 3 weeks later at post-treatment visit (PT) 2.

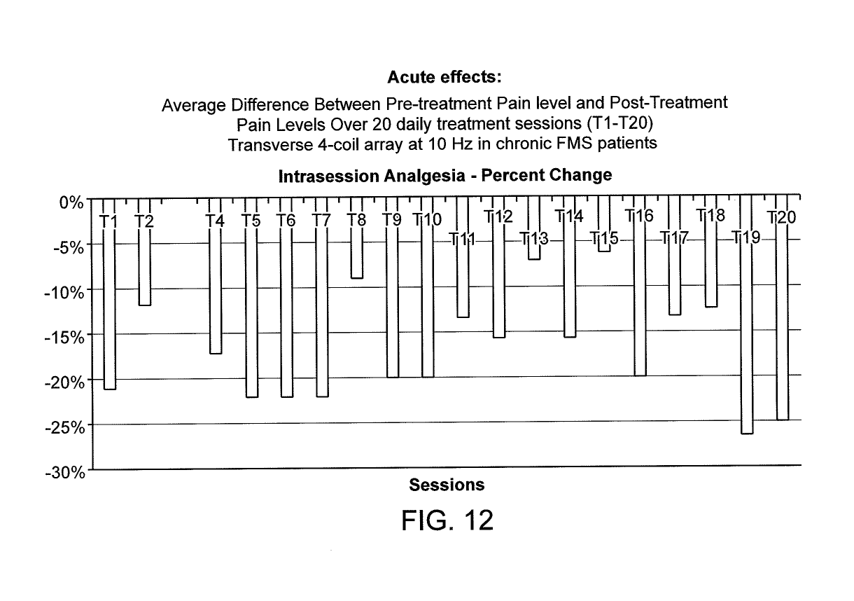

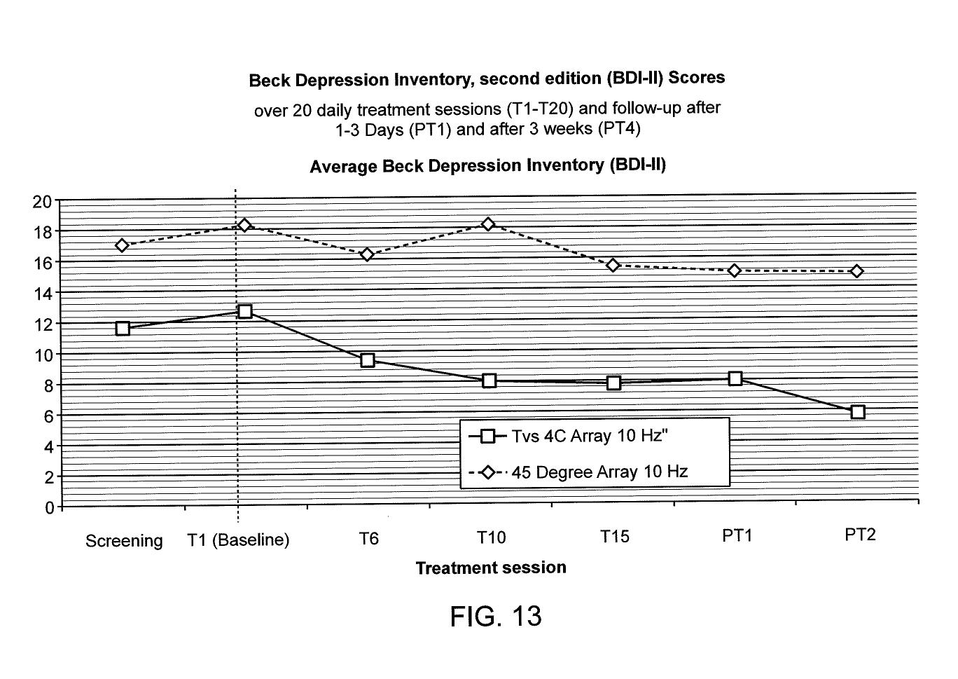

The acute effect of stimulation using this transverse orientation (at 10 Hz) is illustrated in FIG. 12. FIG. 12 is a corollary to the graph shown in FIG. 3, above. While FIG. 3 looked at acute pain reduction when the transverse array was operated at 1 Hz, FIG. 12 shows the acute pain reduction effect of a transverse 4-coil array when operated at 10 Hz, 3000 pulses. Although the data above examined the effect of transverse deep brain stimulation on pain (e.g., acute and chronic pain), other deep brain targets and effects may similarly be achieved using this configuration. Another indication that may be treated includes depression. For example, FIG. 13 documents the antidepressant effect of a 4-coil transverse array operated at 10 Hz as compared with 45-degree multi-coil array as measured by the Beck Depression Inventory, second edition (BDI-II). The transverse array shows a significant reduction in BDI-II scores, but the 45-degree array shows a non-significant reduction in the depression score.

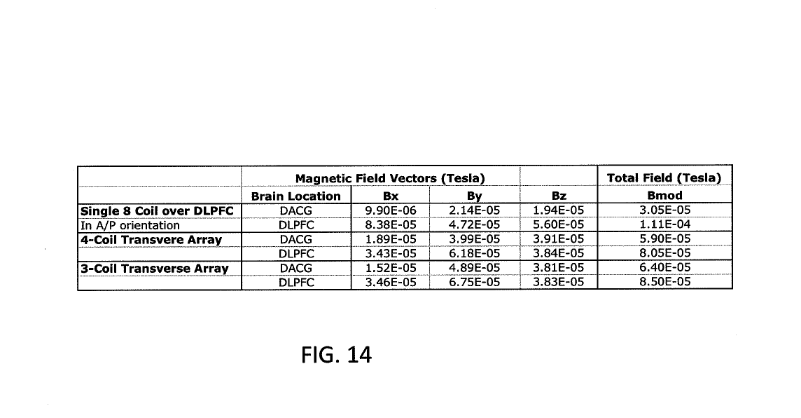

FIG. 14 shows a table of magnetic field vectors associated with transverse coil arrays as compared with a standard single coil in standard (A-P oriented) positioning. Values represent B-field power produced by 1 amp DC simulation input to each coil, and are directly proportional to the values that are produced when powering each coil with an actual TMS pulse generator, but of much smaller magnitude. However for any given power level or type delivered to these coils, the relationship between the magnitudes of the magnetic field at those positions is space will be the same. Specifically, note that the 4-coil transverse array and the 3-coil transverse array both deliver more power to the dorsal anterior cingulate (DACG) than does a standard figure-8 coil placed over the DLPFC. Also note that the three-coil array contains more B-field power in the Y vector, but that the 4-coil array produces almost as much in the Y, and more in Z axis than its 3-coil cousin. Note that the positive or negative values in the B-field vectors (X, Y, Z) are significant in that they add and subtract from one another. However, because the coils used with the present invention are frequently biphasic, these polarities will reverse one or more times during the firing of a pulse from each coil.

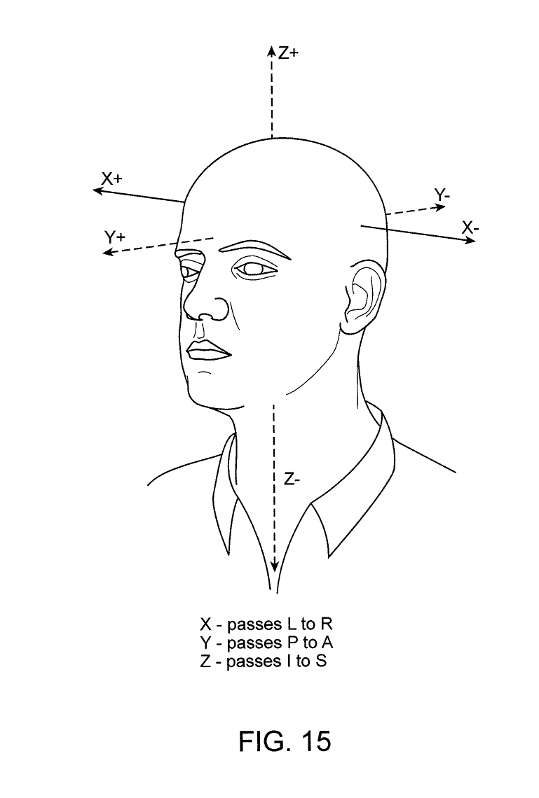

FIG. 15 illustrates the directions of the X, Y and Z coordinates as used in the FIG. 14 table. In summary, X is in the coronal plane, passing from left (negative) to right (positive). Y is in the sagittal plane, passing from posterior (negative) to anterior (positive). Z is in the axial plane, passing from inferior (negative) to superior (positive).

Thus, in general, the methods described herein may be used to apply stimulation (including low-frequency stimulation) using a TMS system to reduce pain via positioning the TMS electromagnet(s). In some variations, the TMS electromagnets may be oriented so that the principle direction of current in the TMS electromagnet is transverse to the AP axis of the subject's head (brain). The principle direction of current in the TMS electromagnet may be the net direction of current, or it may be the direction of current in the geometric center of the TMS electromagnet, particularly in TMS electromagnets having dual coils. Any appropriate TMS electromagnet configuration may be used, including, but not limited to, figure-8 coils that are flat, bent or curved, V-shaped TMS electromagnets, swept-wing or flat-bottomed TMS electromagnets, or the like. Examples of different TMS electromagnet configurations may be found in International Patent Application No. PCT/US2008/075706, Publication No. WO 2009/033192, titled “FOCUSED MAGNETIC FIELDS,” filed Sep. 7, 2008, and in International Patent Application No. PCT/US2010/020324, Publication No. WO 2010/080879, titled “SHAPED COILS FOR TRANSCRANIAL MAGNETIC STIMULATION,” filed on Jan. 7, 2010, each of which is herein incorporated by reference in their entirety.

In some variations, one, some, or all of the TMS electromagnets are oriented transverse to the AP axis of the subject's head when applying low-frequency TMS. In some variations the direction of the net induced current from the plurality of TMS electromagnets is transverse to the AP axis. As used herein, low-frequency TMS may be used synonymously with slow rate or slow rTMS pulse rates, and may be between about 0.5 Hz to about 2 Hz. This low-frequency or slow rate rTMS pulse rates may be contrasted with “fast” rTMS pulse rates (e.g., between about 5-50 Hz). Thus, in some variations, low-frequency TMS may be less than about 5 Hz.

### TMS Systems

In some variations a TMS system may include four TMS electromagnets, three TMS electromagnets, two TMS electromagnets, etc. Variation and examples of such systems that may be used or adapted for use as described herein are provided below. In general, a TMS electromagnet may be referred to as a TMS coil, even though it has multiple “coils” or loops of conductive material used to form the magnetic field.

In one example of a TMS system, the system is a multi-coil device that delivers repetitive transcranial magnetic stimulation (rTMS). The System may consist of pulse generators units, coordinated by a controller (e.g., touch panel controller, TPC), which drive electromagnets, referred to as coils. A positioning device (applicator) may hold the coils in fixed relation to the subject's head and to each other. When activated, the electromagnetic coils stimulate activity within underlying brain targets. To prevent overheating, a circulating system delivers coolant to all coils. In some variations, protection circuits disable the system if the temperature of any one of the stimulating coils exceeds 40° C. This is referred to as a coil overheat event. Following a coil overheat event, the system will not restart automatically, but may be manually restarted when all coils have cooled to 30° C. or less.

A clinician may control the coils through a simple interface on the TPC, which includes controls to perform the functions listed below. The TPC also has password-protected features, such as a non-volatile device log, and a protocol editor that are for use by appropriate personnel only.

In some variations, at the start and/or finish of a treatment, the operator may determine a subject's motor threshold (MT). The operator may then select and run a treatment protocol. The system may dynamically adjust the stimulation power level during the protocol for subject comfort. In use, the subject may sit in a chair for treatment and wear earplugs to protect against the sound of coil discharge. A clinician may also wear ear protection during the subject's treatment. The clinician typically positions the coil used for determining MT relative to the subject's head, and then performs a MT calibration as described in Section IV or V, System Operation. Once positioning and MT are complete, the clinician may establish the treatment power and begin the treatment protocol. There are several parameters used in rTMS treatment, which the clinician can vary in order to optimize the treatment effect of rTMS on the subject's targeted brain structures. The rTMS treatment parameters that determine the rTMS dosage as well as the specific values associated with a protocol may include: Frequency or pulse rate; Treatment power; Pulse train duration; Pulse train rest; Duration of treatment; Minimum treatment time; and Pulses per treatment session.

For example, the frequency or pulse rate may refer to the number of times per second a pulse is delivered. In some variations, the rTMS frequencies are between about 1-10 Hz, e.g., 10 Hz. The treatment power may refer to the maximum summed power for all coils within each array, such as less than about <120% of MT. Pulses may be delivered in sets called trains. The pulse train duration may refer to the amount of time required to deliver each set of pulses, such as <4 seconds within each 30-second period. Pulse train rest may refer to the amount of time between pulse trains, such as at least 26 seconds per 30 second period. The duration of treatment may refer to the amount of time from the delivery of the first pulse to the last pulse, such as approximately 37.5 minutes. The minimum treatment time may be the shortest amount of time from the delivery of the first pulse that can be considered a complete treatment (e.g., 30 minutes). The pulses per treatment session may refer to the total number of pulses delivered per treatment session (e.g., 3000).

A system as described herein may include all of some of: a System Chair; Coil Positioning System (“CPS”); Touch Panel Controller (“TPC”); Electromagnetic Stimulating Coils (“coils”); Stimulator units (“pulse generators”); Cooling system; and Power distribution system.

In some variations, the System Chair provides comfortable positioning for the subject during the procedure. The seat may recline and contain a headrest and a means in which to connect the seat to the CPS.

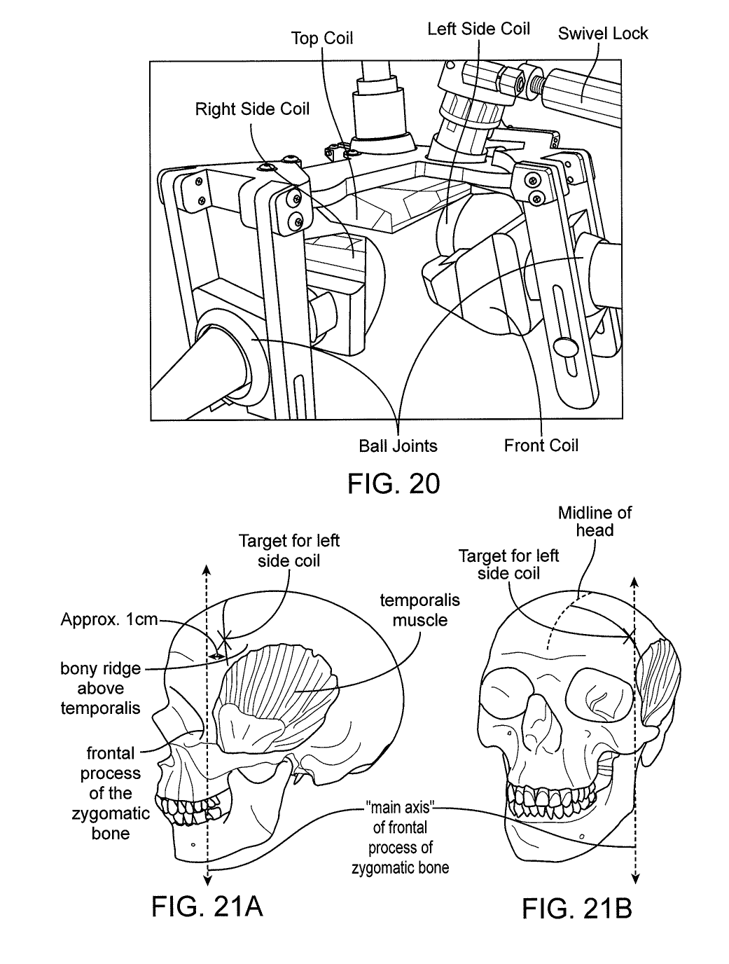

FIG. 20 illustrates one variation of a Coil Positioning System (“CPS”). In this example, the CPS is an applicator that includes a framework and four TMS electromagnet mounts. The CPS show in FIG. 20 is comprised of two articulated arms, with a 4-coil holder, for positioning the coils relative to the subject's head, and physical supports for the pulse generators, power distribution system, TPC, and cooling system. In this example, the CPS mounts each coil holder on an articulated arm for simple positioning. Clamp knobs secure the coils and CPS arms, as shown in FIG. 20. To reposition a coil or CPS arm, loosen the corresponding clamp knob, move the coil so it establishes continuous gentle contact with the subject's head, and re-tighten the clamp knob.

The TPC is a touch screen PC designed to provide a user interface for the clinician in selecting and running a protocol. The TPC also comprises a microcontroller that sends trigger pulses to the pulse generators, measures ambient temperature, provides an audible warning when pulsing is about to begin, and controls the cooling unit. Within each TMS electromagnet (“coil”) is a double metal coil contained within a plastic housing that is positioned at certain anatomic locations on the subject's head. When driven by the stimulator unit, the electromagnetic coil produces a magnetic field, which then results in the depolarization of neurons in predetermined target locations within the subject's brain. The Stimulator units (“pulse generators”) typically provide energy to the coils. The system may also include a cooling subsystem (“cooling system”). The coils may be cooled throughout and between sessions via a circulating system, which delivers coolant to all coils. The power distribution system provides a master power switch, safety circuit breakers, and outlets for the various system components so that the system can be powered from a single circuit.

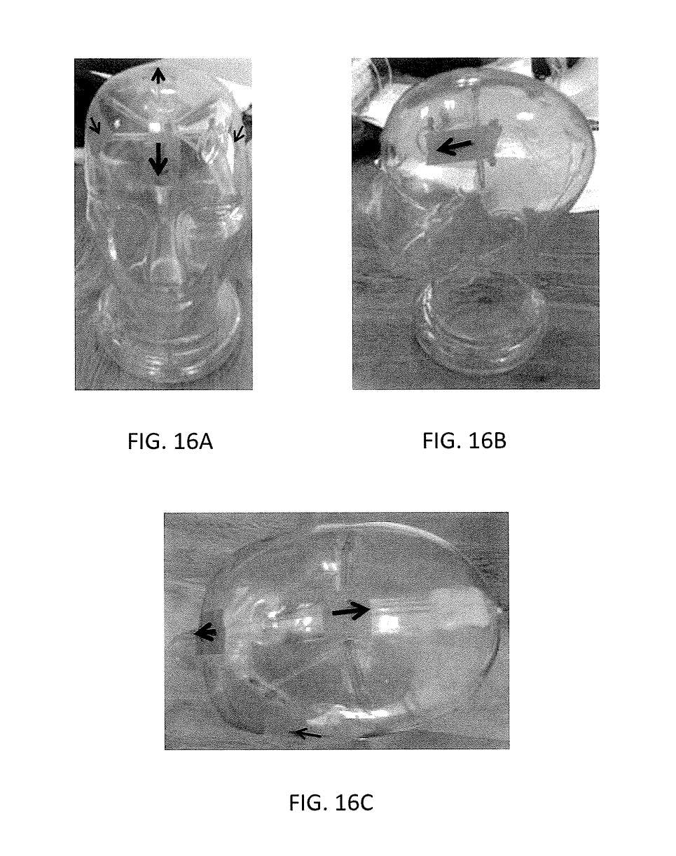

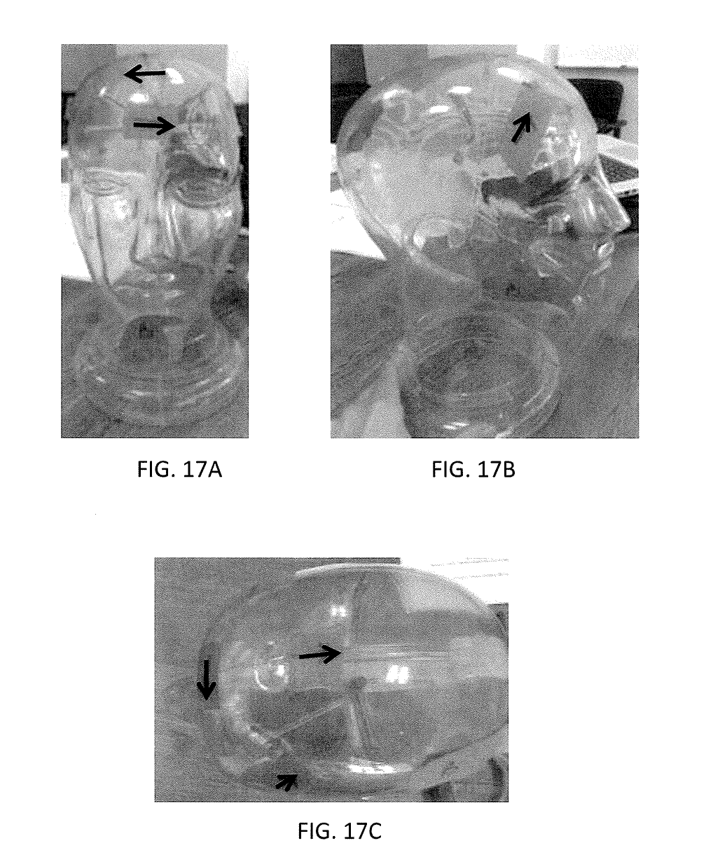

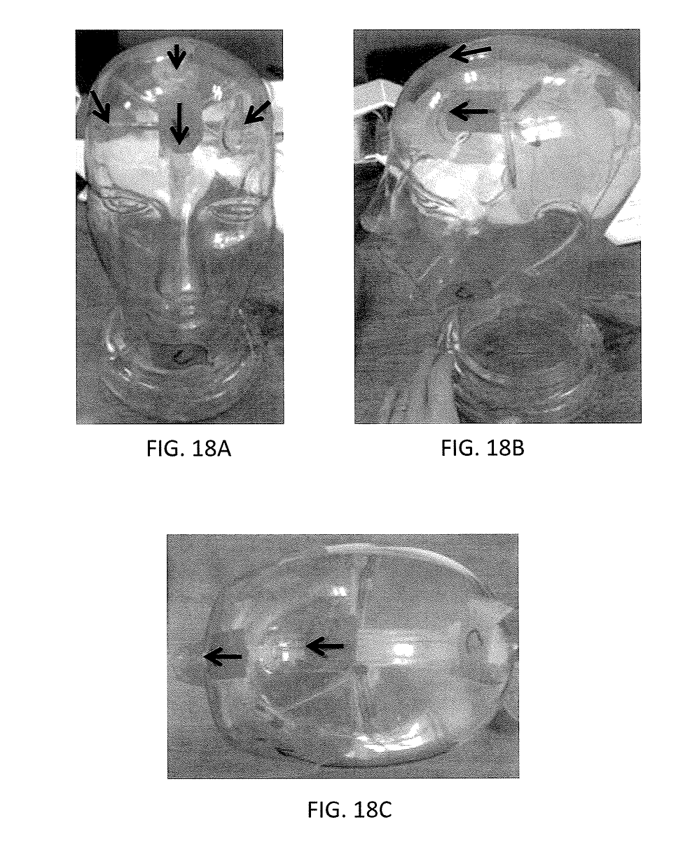

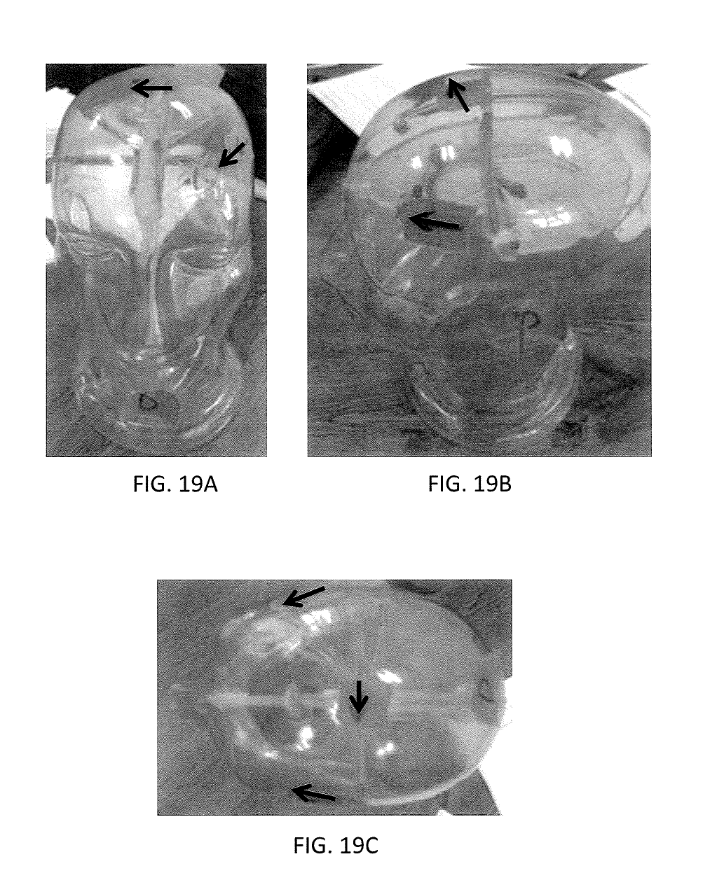

FIGS. 16A-16C, 17A-17C, 18A-18C, and 19A-19C illustrate various placement configurations for TMS electromagnets. FIGS. 16A-16C illustrate a four-TMS electromagnet configuration referred to as the “A” configuration in which a top TMS electromagnet is positioned with the apex of a TMS electromagnet anterior and within a few cm of the Cz point on the subject's head. The front TMS electromagnet is within a few cm (e.g., 1 cm) anterior of the Fz point on the subject's head. Side coils are positioned on the patient's left and right side, respectively between the C3 and F3 (left) and C4 and F4 (right) points. The orientation of the priority direction of current through each TMS electromagnet is indicated by the arrows shown on the model of the head. The overall positions of the TMS electromagnets (top, front, left side, right side) are the same in FIGS. 16A-16C, FIGS. 17A-17C and FIGS. 18A-18C. The configuration shown in FIGS. 17A-17C may be referred to as the “B” configuration (similar to that shown in FIG. 8), and the configuration shown in FIGS. 18A-18C may be referred to as the “C” configuration. FIG. 19A-19C shows a three-coil variation (the “D” configuration) which does not include the front coil shown in FIGS. 16A-18C.

Described below is one example of a method of positioning the TMS electromangets according to configuration such as the A-D configurations illustrated above. In general, positioning of a system as described herein may be performed manually (e.g., by manually positioning each TMS electromagnet) or in an automatic or semi-automatic manner, using an applicator that pre-positions the TMS electromagnets in at least the grossly appropriate manner, though specific adjustments may be made to accommodate the shapes and sizes of different patient's heads.

For example, a four-coil (4 TMS electromagnets) configuration may be formed by surveying for anatomic landmarks of the patient's head. The operator may take note of where each ear is located with respect to the forehead and the top of head, as well as the overall shape of the subject's head. The operator may create a mental image of the anatomic landmarks described below for use in positioning the system coils. With the exception of the top coil location, the anatomic landmarks described may not be discrete spots, but rather describe an area (e.g., a two-centimeter area) where the coil should be placed. For safety considerations, stimulation of the motor cortex at fast rates (e.g., 10 Hz) may be avoided. To avoid the motor cortex, the contact surface of the center of the top coil may be placed forward of the imaginary line which connects the tragus of each ear.

FIGS. 21A and 21B illustrate positions around a skull model. To locate coil position locations, the subject may sit upright in the treatment chair with their back and head away from backrest and headrest. The top coil location may be identified (and placed) first. The top coil may be positioned on the top of the head, directly above the tragus along the midline, as indicated in FIGS. 21A-21B and FIG. 7. The apex (e.g., center) of the top coil may be placed one centimeter anterior (in front of) to that location. The top coil is not placed over motor cortex. If the top coil is inadvertently placed over motor cortex, involuntary movement of the limbs or body will become apparent once pulsing begins. If such movement occurs, reposition the coil further anterior until such movement disappears.

When positioning the coils, care should be taken to place the coil above the temporalis muscle. Placing the coil over, or too close to, the temporalis muscle may result in excessive discomfort and/or involuntary jaw movement. When manually positioning the side coils, begin by locating the temporalis muscle that runs vertically between the ear and the temple. To help locate this muscle, ask the subject to clench his or her jaw several times—the movement of the temporalis muscle can then be seen and felt.

Now move your hand upward along the muscle to locate the bony ridge at which the muscle movement is no longer be seen or felt. This is the origin of the temporalis muscle. It is generally located in line with the outer edge of the eye socket. The left side coil may be placed at a height of about one fourth (25%) to one third (33%) of the distance along the surface of the skull between the bony ridge and the midline of the skull. To determine how far forward or backward of the height landmark the coil should be placed, palpate the frontal process of the zygomatic bone, just lateral to the subject's left eye. Imagine a line passing through this portion of the bone from the floor to the ceiling. Now imagine a parallel line 1 cm behind it. The point at which that second line passes the 25-35% mark defined above is the target for each side coil. In the example of FIGS. 16A-18C, side coils can be placed on both sides of the head, as close to the designated position “X” shown in FIG. 10 above. The front coil may be located anterior to the top coil. The front coil body may be separated from the top coil body by at least one centimeter. As shown in FIG. 7, the shaded area may indicate the general area over where the “hotspots” of the right and left coils in two-coil configuration should be placed. The location of the coil “hotspot” may be adjusted within approximately a two-centimeter radius from the identified target point.

The coils may then be placed against the subject's head. All four coils may maintain gentle but firm contact with subject's head. The patient may be asked if each of the four coil surfaces can be felt touching the scalp. Once the four coils are placed in the appropriate locations around the subject's head, the treatment can commence.

### Example 1

### Steerable Electrical Currents Using Multi-Coil rTMS: Clinical Effects

This example describes the development and early clinical experience with a rTMS device having multiple coils (e.g., 4 coils) such as the ones described above. The system is designed to produce preferential stimulation of brain regions beneath the cortical surface via steerable electrical currents produced by the reconfiguration of the magnets which can accommodate the directional preference of specific brain structures.

Both acute pain and chronic pain have been linked to abnormal activity patterns in the dorsal anterior cingulate gyrus. At one time, surgical lesioning of the cingulate bundles was a commonly practiced treatment for severe, intractable pain. Accordingly, we hypothesized that: (1) induced currents can be steered to depth using a multi-coil approach; (2) multi-coil rTMS can preferentially modulate the dorsal anterior cingulate gyms while relatively sparing the cortical surface; (3) analgesia will be produced; and (4) the same approach will also be effective in the chronic pain of fibromyalgia.

### Methods

PET/Acute Pain Study:

Four custom-shaped TMS electromagnets were closely positioned as illustrated and discussed above, and trained upon the dorsal anterior cingulate gyms, in four different layouts, and referred to as Configurations A, B, C and D, and illustrated in FIGS. 16A-16C, 17A-17C, 18A-18C, and 19A-19C, respectively. The coils were designed to pulse in synchrony, and intended to produce unique magnetic field shapes, and consequently steered induced electrical current. The device was tested on 19 volunteers with thermally induced (acute) pain. Each subject received a blocked sequence of sham, then real multi-coil rTMS at 1 Hz for 30 minutes. ¹⁵O-H₂O PET imaging was acquired concurrently with minute-by-minute numerical rating scale following each stimulation.

Subsequently, using computerized finite-element analysis (FEA), simulations were conducted for each specific coil configuration, and the power levels received by each individual subject. Peak magnetic field magnitude and direction were thus calculated for a series of volumes of interest in the brain. The device was then applied to 45 patients with fibromyalgia pain, in the context off 20 sessions. Four arms of the study were: Configurations A and B (n=17); 1 Hz sham (n=9); 10 Hz Configuration E open label (n=4); and Configuration B open label 10 Hz (n=5).

Brain imaging of induced pain stimulus in sham and real treatments showed cingulate modulation of the effect being studied. A volume-of interest analysis on regions of the dorsal anterior cingulate gyrus and of the overlying medial prefrontal cortex were compared for response to pain and to real versus sham multi-coil rTMS, and showed a significant difference between sham and treatment.

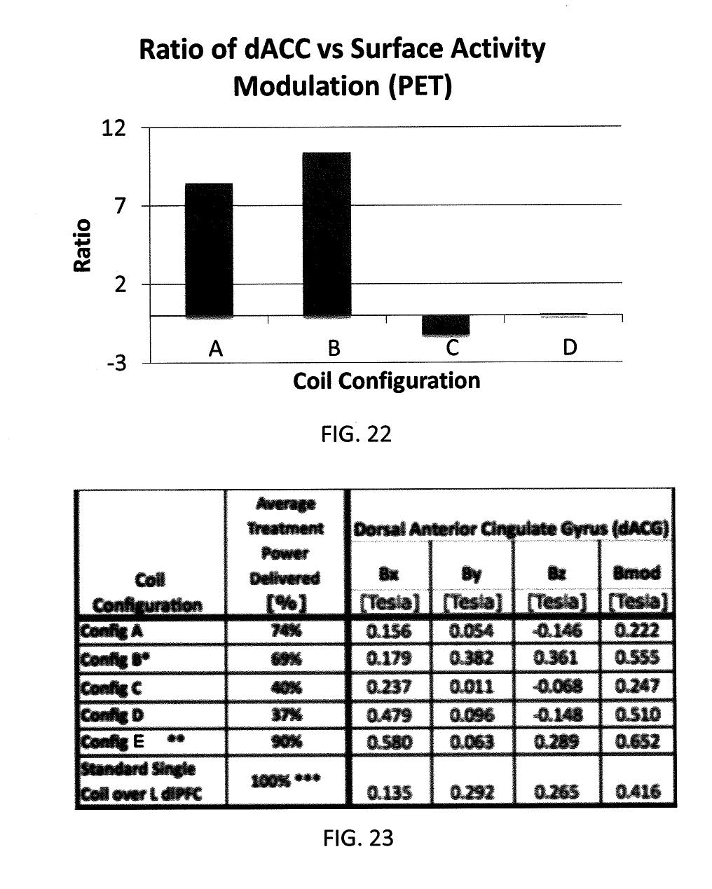

The greatest effect on suppression of pain (analgesia) was seen with configuration B. Configuration B operated at 1 Hz reduced anterior cingulate activity in tandem with an average pain decrease of 48% over sham, and up to 90% decrease. Other configurations were less analgesic, with one configuration (configuration C) increasing pain scores by 32%. Significantly, in configurations A and B, stimulation-induced changes in perfusion at the dACG exceeded the changes on the cortical surface. This is summarized in FIG. 22. In FIG. 22, differential effects of four magnetic coil configurations (A-D) on PET-measured cortical activation in deep vs. surface cerebral cortices are illustrated. Bars represent the ratio of the average impact of magnetic field pulses on dACG cortical activity to the impact on the surface mPFC. Configuration “A” and “B,” both of which produced analgesia in volunteers, demonstrated a much greater effect on deep vs. surface cortical activity. As illustrated in FIG. 22, configuration B is the most selective for cingulate suppression, showing greater suppression of the cingulate more than suppression of overlaying cortical activity by PET.

In both the acute pain study and the fibromyalgia study, power applied to dACG significantly correlates with treatment-induced analgesia (treatment—placebo) after 4 week of treatment. Also, as in the acute pain study, there was a significant correlation between calculated peak magnetic field strength applied to the dACG and analgesic effect in fibromyalgia patients.

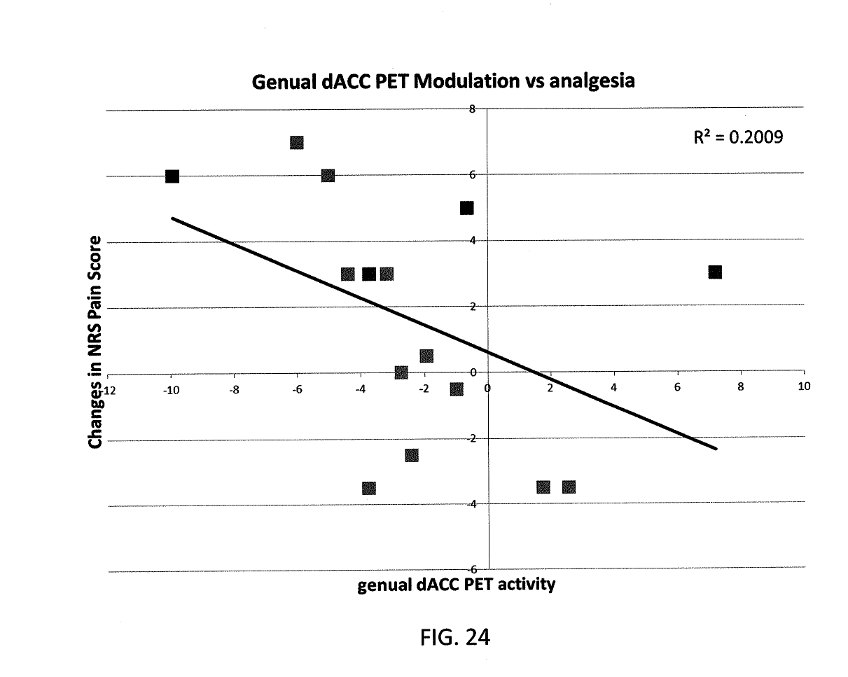

In addition, FEA simulation demonstrated the change in peak magnetic field magnitude and direction at each volume of interest, thereby simulating the system's steerability at depth. This is illustrated in FIG. 23. There is a rough correlation between the change in reported pain and the genual PET activity. Further, there was a significant (p<0.05) correlation between multi-coil TMS modulated perfusion via 0-15 of the rostral-most (genual) dACG and analgesia in volunteers, as illustrated in FIG. 24. Out of seven regions of interest examined (4 surface, 3 deep), only the genual dACG demonstrated a significant correlation between degree of suppression of activity and analgesia.

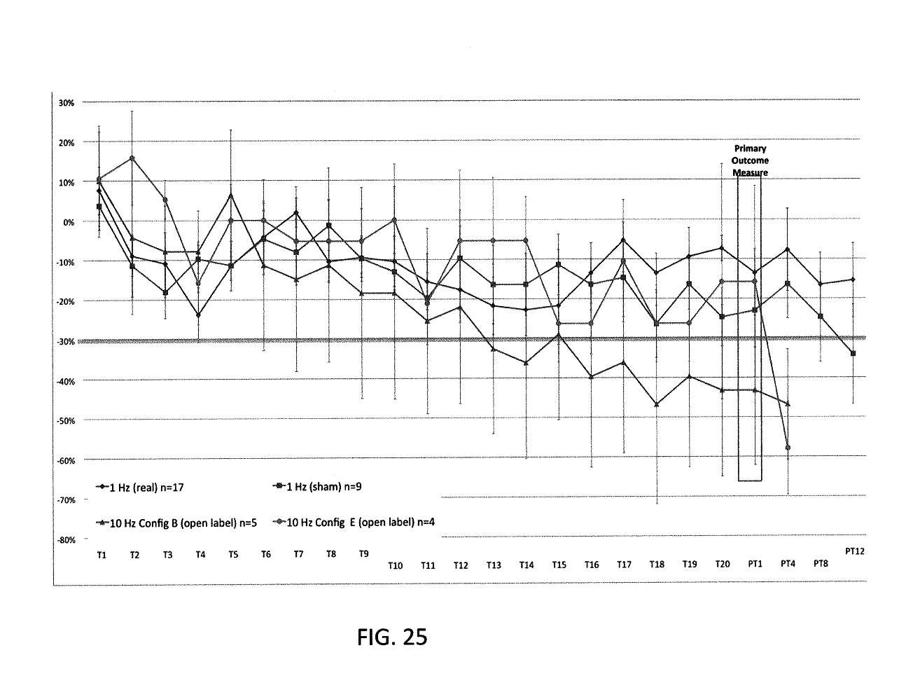

In the fibromyalgia study, the TMS electromagnet arrays operated at 1 Hz were not effective over sham. However when treated with Configuration B at 10 Hz (n=5), the results showed a mean change in pain scores of −43%, which was maintained for at least 4 weeks post-treatment. Changes in the other arms did not exceed that of the sham group (−23%) at the first post-treatment visit, but a market post-treatment effect was noted in one arm. This is illustrated in FIG. 25. The conclusion of this study demonstrates that multi-Coil rTMS can steer current-inducing magnetic fields within the brain. Further, the systems can be used to produce strong analgesia in acute pain by using low-frequency pulse rates. Specific effects are highly dependent upon coil configuration, and TMS electromagnet configuration and power may dictate field shape with respect to brain anatomy, including depth of penetration. Configurations having the best analgesic properties delivered the highest magnetic field dose to the dorsal anterior cingulate. Further, multi-coil rTMS to cortex and cingulate effectively reduces (chronic) fibromyalgia pain, but worked best a rapid pulse rates (e.g., 10 Hz).

In general, the methods and systems described herein may be used to treat pain (and adapted for treatment of pain), including the pain of fibromyalgia. However, it should be clear that these methods and system may also be used to treat other disorders, particularly those whose neural circuits overlap with the pain “circuit” (e.g., including the dACG). For example, a general method of treating a patient may include positioning the TMS electromagnets as described herein, including positioning a top TMS electromagnet with an apex of the TMS electromagnet between about a Cz and Fz location on the patient's head; positioning a front TMS electromagnet with an apex of the TMS electromagnet between about an Fz and Fpz location on the patient's head; and applying stimulation from the top and front TMS electromagnets to the dorsal anterior cingulate gyrus, wherein the Cz, Fz and Fpz locations are determined using a standard 10-20 system for scalp electrode placement. Examples may include methods and systems for treating depression.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached” or “coupled” to another feature or element, it can be directly connected, attached or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of non-invasively treating pain by the application of Transcranial Magnetic Stimulation (TMS) using multiple TMS electromagnets to preferentially stimulate a patient's dorsal anterior cingulate gyrus relative to cortical brain regions, the method comprising:
positioning a top TMS electromagnet with an apex of the TMS electromagnet between about a Cz and Fz location on the patient's head;
positioning a front TMS electromagnet with an apex of the TMS electromagnet between about an Fz and Fpz location on the patient's head; and
modulating pain levels by applying stimulation from the top and front TMS electromagnets to the dorsal anterior cingulate gyrus,
wherein the Cz, Fz and Fpz locations are determined using a standard 10-20 system for scalp electrode placement.

2. The method of claim 1, further comprising positioning an apex of a right side TMS electromagnet on the right side of the patient's head and positioning an apex of a left side TMS electromagnet on the left side of the patient's head.

3. The method of claim 1, further comprising positioning an apex of a right side TMS electromagnet between a C4 and Fp2 locations on the patient's head, wherein the C4 and Fp2 locations are determined using a standard 10-20 system for scalp electrode placement.

4. The method of claim 1, further comprising positioning an apex of a right side TMS electromagnet between a Fp2 and F8 locations on the patient's head, wherein the Fp2 and F8 locations are determined using a standard 10-20 system for scalp electrode placement.

5. The method of claim 1, further comprising positioning an apex of a left side TMS electromagnet between an C3 and Fp1 locations on the patient's head wherein the C3 and Fp1 locations are determined using a standard 10-20 system for scalp electrode placement.

6. The method of claim 1, further comprising positioning an apex of a left side TMS electromagnet between an Fp1 and F7 locations on the patient's head wherein the Fp1 and F7 locations are determined using a standard 10-20 system for scalp electrode placement.

7. The method of claim 1, wherein the top TMS electromagnet is positioned within about 2.5 cm anterior of Cz.

8. The method of claim 1, wherein the front TMS electromagnet is positioned within about 2 cm anterior to Fz.

9. The method of claim 1, wherein the front and top TMS electromagnet comprises a bent figure-8 TMS electromagnet.

10. The method of claim 1, wherein the top TMS electromagnet comprises a swept-wing TMS coil.

11. The method of claim 1, wherein modulating pain levels by applying stimulation from the top and front TMS electromagnets to the dorsal anterior cingulate gyms comprises applying stimulation at a frequency of stimulation from the front and top TMS electromagnets that is above about 5 Hz.

12. The method of claim 1, wherein modulating pain levels comprises reducing pain by applying stimulation from the top and front TMS electromagnets to the dorsal anterior cingulate gyms.

13. The method of claim 1, wherein modulating pain levels comprises reducing the pain of fibromyalgia.

14. A method of non-invasively treating pain by the application of Transcranial Magnetic Stimulation (TMS) using multiple TMS electromagnets to preferentially stimulate a patient's dorsal anterior cingulate gyms relative to cortical brain regions, the method comprising:
positioning a top TMS coil anterior to a Cz location on the patient's head so that the principle direction of electrical current induced by the electromagnet is perpendicular to the anterior-posterior axis of the patient's head;
positioning a front TMS coil anterior to an Fz location on the patient's head;
positioning a left side coil on the left side of the patient's head;
positioning a right side coil on the right side of the patient's head; and
reducing pain levels by applying stimulation from the TMS electromagnets to the dorsal anterior cingulate gyrus,
wherein the Cz, Fz, C3, C4, F3 and F4 locations are determined using a standard 10-20 system for scalp electrode placement.

15. A Transcranial Magnetic Stimulation multi-electromagnet applicator configured to be positioned over a patient's head for non-invasively treating pain by the application of Transcranial Magnetic Stimulation (TMS) using multiple TMS electromagnets to preferentially stimulate a patient's dorsal anterior cingulate gyrus relative to cortical brain regions, the device comprising:
a framework comprising a first mount, a second mount, a left side mount, and a right side mount, wherein the framework holds a plurality of TMS electromagnets in a predetermined arrangement around the patient's head so that when the framework is positioned over the patient's head, a first TMS electromagnet is between about a Cz and Fz location on the patient's head, a second TMS electromagnet is between about an Fz and Fpz location on the patient's head, a left side TMS electromagnets is on the left side of the patient's head, and a right side TMS electromagnet is on the right side of the patient's head; and
the first mount, second mount, left side mount, and right side mount are each configured to secure a TMS electromagnet to the framework and are further configured to allow adjustment of the angle of the TMS electromagnet relative to the framework, and to allow adjustment of a radial distance of the TMS electromagnet from the frame and toward the surface of the patient's head when the framework is positioned over the patient's head,
wherein the Cz, Fz and Fpz locations are determined using a standard 10-20 system for scalp electrode placement.

16. The device of claim 15, further wherein the framework comprises a hinged region configured to move one or more of the first mount, second mount, left side mount, and right side mount out of the predetermined arrangement so that the device can be positioned over the patient's head.

17. The device of claim 15, wherein first mount, second mount, left side mount, and right side mount are each configured to hold a TMS electromagnet so that the TMS electromagnet is pivotable about a contact point with the patient's head when the framework is positioned over the patient's head.

18. The device of claim 15, wherein the first mount, second mount, left side mount, and right side mount each comprise a ball joint.

19. The device of claim 15, further comprising a top TMS electromagnet within the first mount, a front TMS electromagnet within the second mount, a left side TMS electromagnet within the left side mount, and a right side TMS electromagnet within the right side mount.

20. The device of claim 15, wherein the top TMS electromagnet, front TMS electromagnet, left side TMS electromagnet, and right side TMS electromagnet are all bent TMS coils.

21. The device of claim 15, wherein the framework is configured so that when the framework is positioned over the patient's head, the left side TMS electromagnet is between the C3 and Fp1 locations, wherein the C3 and Fp1 locations are determined using a standard 10-20 system for scalp electrode placement.

22. The device of claim 15, wherein the framework is configured so that when the framework is positioned over the patient's head, the right side TMS electromagnet is between the C4 and Fp2 locations, wherein the C4 and Fp2 locations are determined using a standard 10-20 system for scalp electrode placement.

23. A Transcranial Magnetic Stimulation multi-electromagnet applicator configured to be positioned over a patient's head for non-invasively treating pain by the application of Transcranial Magnetic Stimulation (TMS) using multiple TMS electromagnets to preferentially stimulate a patient's dorsal anterior cingulate gyrus relative to cortical brain regions, the device comprising:
a top mount configured to hold a top TMS electromagnet;
a front mount configured to hold a front TMS electromagnet;
a left side mount configured to hold a left side TMS electromagnet;
a right side mount configured to hold a right side TMS electromagnet; and
a framework holding the top mount, front mount, left side mount, and right side mount in a predetermined configuration so that when the device is positioned over the patient's head, the top TMS electromagnet is positioned between about a Cz and Fz location on the patient's head, the front TMS electromagnet is between about an Fz and Fpz location on the patient's head, the left side TMS electromagnets is on the left side of the patient's head, and the right side TMS electromagnet is on the right side of the patient's head;
wherein each mount of the top mount, front mount, left side mount and right side mount allow adjustment of the angle and radial distance of a TMS electromagnet held by each mount relative to the framework,
wherein the Cz, Fz and Fpz locations are determined using a standard 10-20 system for scalp electrode placement.
